(19) United States
(12) Patent Application Publication
(10) Pub. No.: US 2023/0173295 A1
(43) Pub. Date: Jun. 8, 2023

(54) Systems, Devices, and Methods for Gamma Entrainment using Sensory Stimuli to Alleviate Cognitive Deficits and/or Neuroinflammation Induced by Chemotherapy Agents

(71) Applicants: Li-Huei TSAI, Cambridge, MA (US); Thomas Aquinas KIM, Cambridge, MA (US); Massachusetts Institute of Technology, Cambridge, MA (US)

(72) Inventors: Thomas Aquinas Kim, Cambridge, MA (US); Li-Huei Tsai, Cambridge, MA (US)

(73) Assignee: Massachusetts Institute of Technology, Cambridge, MA (US)

(21) Appl. No.: 17/996,855

(22) PCT Filed: Apr. 23, 2021
(86) PCT No.: PCT/US2021/028776

§ 371 Date: Oct. 21, 2022

Related U.S. Application Data
(60) Provisional application No. 63/014,300, filed on Apr. 23, 2020.

(51) Int. Cl. A61N 5/06; A61K 51/04

(52) U.S. Cl. CPC A61N 5/06; A61K 51/0474; A61K 51/0459; A61N 2007/0021

(57) ABSTRACT

A method of treating cognitive impairment associated with chemotherapy treatment in a subject in need thereof includes on-invasively delivering a combined stimulus to the subject to invoke gamma entrainment in a brain of the subject. The combined stimulus includes an auditory stimulus having a frequency of from about 20 Hz to about 60 Hz, and a visual stimulus having a frequency of from about 20 Hz to about 60 Hz.

### CROSS-REFERENCE TO RELATED APPLICATIONS

This application claims priority to U.S. Provisional Application No. 63/014,300 filed Apr. 23, 2020 and titled “SYSTEMS, DEVICES, AND METHODS FOR GAMMA ENTRAINMENT USING SENSORY STIMULI TO ALLEVIATE COGNITIVE DEFICITS AND/OR NEUROINFLAMMATION INDUCED BY CHEMOTHERAPY AGENTS”, the entire disclosure of which is incorporated herein by reference.

### BACKGROUND

Chemo brain is a common term used by cancer patients and/or survivors to describe thinking, memory, and/or other cognitive problems that can occur during and after chemotherapy/chemotherapy treatment. Chemo brain is also sometimes called chemo fog, cancer-related cognitive impairment, cancer-related cognitive change, post-chemotherapy cognitive impairment, or cognitive dysfunction, among others. There is currently no known way to prevent the cognitive changes that cause chemo brain, and the causes of chemo brain are still undetermined.

Patients with chemo brain can suffer from a wide range of cognitive impairments such as memory lapses, lack of focus, shorted attention spans, trouble with memory recall, multi-tasking, learning, organization, reduced speech ability, and/or the like.

### SUMMARY

In some aspects, a method of treating cognitive impairment associated with chemotherapy treatment in a subject in need thereof includes non-invasively delivering a combined stimulus to the subject to invoke gamma entrainment in a brain of the subject. The combined stimulus includes an auditory stimulus having a frequency of from about 20 Hz to about 60 Hz, and a visual stimulus having a frequency of from about 20 Hz to about 60 Hz.

In some aspects, a method of treating cognitive impairment associated with chemotherapy treatment in a subject in need thereof includes delivering a stimulus to the subject to invoke gamma entrainment in a brain of the subject, the stimulus having a frequency of from about 20 Hz to about 60 Hz.

In some aspects, a method of reducing neuroinflammation in a brain region of a subject, the neuroinflammation associated with chemotherapy treatment in the subject in need thereof, includes delivering a stimulus to the subject to invoke gamma entrainment in a brain of the subject, the stimulus having a frequency of from about 20 Hz to about 60 Hz.

In some aspects, a method of reducing deoxyribonucleic acid (DNA) damage in a brain region of a subject, the DNA damage associated with chemotherapy treatment in the subject in need thereof, includes delivering a stimulus to the subject to invoke gamma entrainment in a brain of the subject, the stimulus having a frequency of from about 20 Hz to about 60 Hz.

In some aspects, a method of at least partially reversing an enlargement in ventricle size in a brain region of a subject, the enlargement in ventricle size associated with chemotherapy treatment in the subject in need thereof, includes delivering a stimulus to the subject to invoke gamma entrainment in a brain of the subject, the stimulus having a frequency of from about 20 Hz to about 60 Hz.

In some aspects, a method of increasing generation of at least one of oligodendrocytes or oligodendrocyte precursor cells in a brain region of a subject includes delivering a stimulus to the subject to invoke gamma entrainment in a brain of the subject, the stimulus having a frequency of from about 20 Hz to about 60 Hz.

All combinations of the foregoing concepts and additional concepts are discussed in greater detail below (provided such concepts are not mutually inconsistent) and are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The patent or application file contains at least one drawing executed in color. Copies of this patent or patent application publication with color drawing(s) will be provided by the Office upon request and payment of the necessary fee.

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

Unless expressly indicated otherwise, all graphs/plots are displayed as mean±SEM.

FIGS. 1A-1I illustrate that Gamma ENtrainment Using Sensory stimulus (GENUS) mitigated demyelination in chemo brain model animals. FIG. 1A illustrates a protocol for inducing a chemo brain mouse model with methotrexate (MTX).

FIG. 1B illustrates a protocol for inducing a chemo brain mouse model with cisplatin.

FIG. 1C is a plot illustrating that Cisplatin treated animals showed up to 40% body weight loss compared to their original body weight. GENUS did not affect the body weight. PBS with no sensory stimulus (PBS NS), n=8; PBS with 40 Hz audio+visual sensory stimulus (PBS S), n=8; cisplatin with no sensory stimulus (Cis NS), n=7; cisplatin with 40 Hz audio+visual sensory stimulus (Cis S), n=7; 3-way repeated measure ANOVA, effect of cisplatin, F_{1, 26}=377.6, ****p<0.0001, effect of GENUS F_{1, 26}=0.7193, p=0.4041.

FIG. 1D are images of Iba1 (green) staining in the corpus callosum (cc) for PBS NS (1^{st} panel), PBS S (2^{nd} panel), Cis NS (3^{rd} panel), and Cis S (4^{th} panel).

FIG. 1E is a plot illustrating that Iba1 signal in Cis NS corpus callosum was significantly higher than Cis S. PBS NS, n=13 slices from 7 animals; PBS S=12 slices from 6 animals; MTX NS, n=7 slices from 4 animals; MTX S, n=8 slices from 5 animals; 2-way ANOVA, interaction between cisplatin and GENUS F_{1, 36}=5.482, *p<0.05, Tukey's multiple comparisons test, Cis NS vs. Cis S *p<0.05.

FIGS. 1F, 1G illustrate that 40 Hz combined audio+visual stimulation mitigated demyelination in cisplatin induced chemo brain animal model. FIG. 1F illustrates Myelin (Myelin basic protein (MBP), green), OPCs and oligodendrocytes (Olig2, red), nuclei (Hoechst, blue) staining of cingulate cortex area for PBS NS (1^{st} panel), PBS S (2^{nd} panel), Cis NS (3^{rd} panel), and Cis S (4^{th} panel).

FIG. 1G is a plot illustrating quantification of cingulate cortex area covered by MBP protein. PBS NS, n=10 slices from 5 animals; PBS S, n=10 slices from 5 animals; Cis NS, n=10 slices from 5 animals; Cis S, n=12 slices from 6 animals; 2-way ANOVA, Tukey's multiple comparisons test, interaction between cisplatin and GENUS F_{1, 38}=4.951, *p<0.05, PBS NS vs Cis NS, *p<0.05; PBS S vs. Cis NS, *p<0.05; Cis NS vs. Cis S, *p<0.01.

FIG. 1H is a plot illustrating that the number of Olig2⁺ cells in grey matter (ACC) did not differ between groups. PBS NS, n=4 slices from 4 animals; PBS S, n=4 slices from 4 animals; Cis NS, n=4 slices from 4 animals; Cis S, n=5 slices from 5 animals; 2-way ANOVA, Tukey's multiple comparisons test, interaction between cisplatin and GENUS F_{1, 13}=1.726, p=0.2116.

FIG. 1I is a plot illustrating that the number of Olig2⁺ cells in white matter (corpus callosum) did increase after GENUS treatment. PBS NS, n=4 slices from 4 animals; PBS S, n=4 slices from 4 animals; Cis NS, n=4 slices from 4 animals; Cis S, n=4 slices from 4 animals; 2-way ANOVA, Tukey's multiple comparisons test, effect of GENUS F_{1, 12}=5.258, *p<0.05.

FIG. 1J is a plot illustrating that neither MTX nor GENUS altered swimming speed of animals during a Morris Water Maze (MWM) test. PBS NS, n=8; PBS S, n=8; MTX NS, n=7; MTX S, n=6; 3-way repeated measure ANOVA, interaction between MTX and GENUS, F_{1, 25}=0.1428, p=0.7087, 1 animal from MTX S group was excluded from the analysis because of floating behavior.

FIG. 1K is a plot illustrating that neither MTX nor GENUS altered learning curve during the MWM test. PBS NS, n=8; PBS S, n=8; MTX NS, n=7; MTX S, n=6; 3-way repeated measure ANOVA, interaction between MTX and GENUS F_{1, 100}=2.343, p=0.1290.

FIG. 1L is a plot illustrating that neither MTX nor GENUS altered learning curve during the MWM test. PBS NS, n=8; PBS S, n=8; MTX NS, n=7; MTX S, n=6; 2-way ANOVA, interaction between MTX and GENUS F_{1, 25}=2.814, p=0.0959.

FIGS. 2A-2E illustrate that GENUS protects brain cells from DNA damage and neuroinflammation. FIG. 2A illustrates γH2AX (green) and Hoechst (blue) staining in prefrontal cortex for PBS NS (top-left panel), PBS S (bottom-left panel), Cis NS (top-right panel), and Cis S (bottom-right panel).

FIG. 2B is a plot illustrating that γH2AX⁺ cell number was significantly increased after cisplatin treatment, but Cis S group had significantly lower number of γH2AX⁺ cells compared to Cis NS group, PBS NS, n=7 slices from 7 animals; PBS S, n=5 slices from 5 animals; cisplatin NS, n=6 slices from 6 animals; cisplatin S, n=6 slices from 6 animals. 2-way ANOVA, interaction between cisplatin and GENUS F_{1,20}=6.870 *p<0.05, Tukey's multiple comparisons test, PBS NS vs. Cis NS ****p<0.0001, PBS S vs. Cis NS ****p<0.0001, Cis NS vs. Cis S **p<0.01. 1 animal from PBS S groups was excluded as an outlier, ROUT test, Q=1%, outlier value=892.

FIG. 2C illustrates Iba1 (red), GFAP (white) and Hoechst (blue) staining of hippocampal CA1 region for PBS NS (top-left panel), PBS S (bottom-left panel), Cis NS (top-right panel), and Cis S (bottom-right panel).

FIG. 2D is a plot illustrating that CA1 of Cis NS animals showed astrogliosis, while Cis S animals showed comparable level of area covered by GFAP signal PBS NS, n=13 slices from 7 animals; PBS S, n=10 slices from 7 animals; cisplatin NS, n=10 slices from 6 animals; cisplatin S, n=16 slices from 8 animals. 2-way ANOVA, interaction between cisplatin and GENUS F_{1,45}=17.81 ***p<0.0001, Tukey's multiple comparisons test, PBS NS vs. Cis NS ***p<0.001, PBS S vs. Cis NS *p<0.05, Cis NS vs. Cis S ***p<0.001.

FIG. 2E is a plot illustrating that CA1 of Cis NS animals showed increased number of microglia, while Cis S animals showed comparable level of area covered by GFAP signal PBS NS, n=12 slices from 6 animals; PBS S, n=9 slices from 7 animals; cisplatin NS, n=10 slices from 6 animals; cisplatin S, n=13 slices from 6 animals. 2-way ANOVA, Tukey's multiple comparisons test, PBS NS vs. Cis NS *p<0.05, PBS S vs. Cis NS **p<0.01, Cis NS vs. Cis S *p<0.05. All graphs displayed as mean±SEM.

FIGS. 3A-3L illustrate that GENUS improved cognitive function in chemo brain animals. FIG. 3A is a plot illustrating that Cisplatin treated animals showed impaired locomotion during OFT. PBS NS, n=8; PBS S, n=8; Cis NS, n=7; Cis S, n=7; 2-way ANOVA, effect of cisplatin F_{1, 26}=73.77, ****p<0.0001.

FIG. 3B is a plot illustrating that GENUS, but not cisplatin treated animals, spent more time in the center area during OFT. PBS NS, n=8; PBS S, n=8; Cis NS, n=7; Cis S, n=7; 2-way ANOVA, effect of GENUS F_{1, 26}=4.483, *p<0.05, effect of cisplatin F_{1, 26}=1.663, p=0.2085. Graph displayed as mean±SEM.

FIG. 3C is a plot illustrating that GENUS mitigated cognitive impairment caused by cisplatin treatment in novel object recognition (NOR) test. PBS NS, n=8; PBS S, n=7; Cis NS, n=8; Cis S, n=8; 2-way ANOVA, interaction between MTX and GENUS, F_{1, 27}=5.991, *p<0.05, Tukey's multiple comparisons test, PBS NS vs. Cis NS *p<0.05, PBS S vs. Cis NS *p<0.05, Cis NS vs. Cis S **p<0.01.

FIGS. 3D-3F are plots illustrating that GENUS rescued cognitive impairment caused by cisplatin treatment in puzzle box test. FIG. 3D is a plot illustrating an average of three trials of the easy task, PBS NS, n=8; PBS S, n=8; Cis NS, n=7; Cis S, n=7; 2-way ANOVA, effect of cisplatin F_{1, 26}=28.42, ****p<0.0001.

FIG. 3E is a plot illustrating an average of three trials of the intermediate task, PBS NS, n=8; PBS S, n=8; Cis NS, n=7; Cis S, n=7; 2-way ANOVA, effect of cisplatin F_{1, 26}=8.549, **p<0.01, Tukey's multiple comparisons test, PBS NS vs. cisplatin NS, *p<0.05.

FIG. 3F is a plot illustrating an average of two trials of the hard task PBS NS, n=8; PBS S, n=8; Cis NS, n=7; Cis S, n=7; 2-way ANOVA, effect of cisplatin F_{1, 26}=6.747, *p<0.05, effect of GENUS F_{1, 26}=7.201, *p<0.05, Tukey's multiple comparisons test, PBS NS vs. Cis NS *p<0.05, PBS S vs. Cis NS **p<0.01, Cis NS vs. Cis S *p<0.05.

FIG. 3G is a plot illustrating that neither GENUS nor methotrexate (MTX) treatment affected locomotion of animals in OFT. PBS NS, n=8; PBS S, n=8; MTX NS, n=8; MTX S, n=8; 2-way ANOVA, effect of MTX, F_{1, 32}=0.2040, p=0.6546, effect of GENUS, F_{1, 32}=2.224, p=0.1457.

FIG. 3H is a plot illustrating that GENUS but not MTX significantly increased time spent in center area during OFT. PBS NS, n=8; PBS S, n=8; MTX NS, n=8; MTX S, n=8; 2-way ANOVA, effect of GENUS, F_{1, 32}=5.298, *p<0.05.

FIG. 3I is a plot illustrating that GENUS rescued cognitive impairment caused by MTX treatment in novel object recognition test (NOR). PBS NS, n=8; PBS S, n=8; MTX NS, n=7; MTX S, n=7; 2-way ANOVA, interaction between MTX and GENUS, F_{1, 26}=8.409, **p<0.01, Tukey's multiple comparisons test, PBS NS vs. MTX NS **p<0.01, PBS S vs. MTX S *p<0.05, MTX NS vs. MTX S *p<0.01.

FIGS. 3J-3L are plots illustrating that GENUS rescued cognitive impairment caused by MTX treatment in puzzle box test. FIG. 3J is a plot illustrating an average of three trials of easy task, PBS NS, n=8; PBS S, n=8; MTX NS, n=12; MTX S, n=12; 2-way ANOVA, effect of GENUS F_{1, 36}=3.500, p=0.0695, effect of MTX F_{1, 36}=1.740, p=0.1955.

FIG. 3K is a plot illustrating an average of three trials of intermediate task, PBS NS, n=8; PBS S, n=8; MTX NS, n=12, MTX S, n=12; 2-way ANOVA, effect of MTX F_{1, 36}=17.53, ***p<0.001, Tukey's multiple comparisons test, PBS NS vs. MTX NS, **p<0.01, PBS S vs. MTX NS, **p<0.01, PBS NS vs. MTX S, p=0.3025, PBS S vs. MTX S, p=0.0652.

FIG. 3L is a plot illustrating an average of two trials of hard task PBS NS, n=8; PBS S, n=8; MTX NS, n=12; MTX S, n=12; 2-way ANOVA, interaction between MTX and GENUS F_{1, 36}=13.38, ***p<0.001, Tukey's multiple comparisons test, PBS NS vs. MTX NS ***p<0.001, MTX NS vs. MTX S *p<0.05.

FIG. 4A illustrates Olig2 (red) and Hoechst (blue) staining of corpus callosum for PBS NS (top-left panel), PBS S (bottom-left panel), Cis NS (top-right panel), and Cis S (bottom-right panel).

FIG. 4B illustrates Pdgfrα (green), Olig2 (red) and Hoechst (Blue) staining of corpus callosum (cc) for Cis NS (left panel), and Cis S (right panel).

FIG. 4C is a plot illustrating that Cis NS and Cis S animals had comparable number of OPCs in corpus callosum. Cis NS=7 slices from 4 animals, Cis S=4 slices from 2 animals, unpaired t-test p=0.9191.

FIG. 4D is a plot illustrating that Cis S had significantly higher number of oligodendrocytes compared to Cis NS in corpus callosum. Cis NS=7 slices from 4 animals, Cis S=4 slices from 2 animals, unpaired t-test **p<0.01.

FIG. 4E illustrates Pdgfrα (green), Olig2 (red) and Hoechst (Blue) staining of CA1 region for Cis NS (left panel), and Cis S (right panel).

FIG. 4F is a plot illustrating that Cis S had significantly higher number of OPC compared to Cis NS in CA1. Cis NS=7 slices from 4 animals, Cis S=4 slices from 2 animals, unpaired t-test p<0.05.

FIG. 4G is a plot illustrating that Cis NS and Cis S animals had comparable number of oligodendrocytes in CA1. Cis NS=7 slices from 4 animals, Cis S=4 slices from 2 animals, unpaired t-test p=0.6333.

FIG. 5A illustrates Doublecortin (Red) and EdU (white) staining of dentate gyrus for PBS NS (top-left panel), PBS S (bottom-left panel), Cis NS (top-right panel), and Cis S (bottom-right panel). EdU was injected on experiment day 18˜21 (3˜6 days post final cisplatin injection)

FIG. 5B is a plot illustrating that GENUS restored neurogenesis in cisplatin treated animals to comparable level with PBS control groups. PBS NS, n=7 slices from 4 animals; PBS S, n=8 slices from 4 animals; Cis NS, n=8 slices from 4 animals; Cis S, n=7 slices from 4 animals; 2-way ANOVA, effect of GENUS F_{1, 26}=4.397, *p<0.05, effect of cisplatin F_{1, 26}=12.43, **p=0.01, Tukey's multiple comparisons test, PBS S vs. Cis NS **p<0.01.

FIG. 5C illustrates Doublecortin (Red) and EdU (white) staining of dentate gyrus for Cis NS (left panel), and Cis S (right panel). EdU was injected on experiment day 12˜18 (during last 3 days of cisplatin injection).

FIG. 5D is a plot illustrating that GENUS had no effect on neurogenesis during cisplatin treatment. Cis NS, n=8 slices from 4 animals; Cis S, n=8 slices from 4 animals; unpaired t-test, p=0.5601.

FIG. 5E is a plot illustrating that Cis S animals show higher number of Dcx⁺ cells at experimental day 21 (6 days post final cisplatin treatment). Cis NS, n=8 slices from 4 animals; Cis S, n=8 slices from 4 animals; unpaired t-test, *p<0.05.

FIG. 6A are whole slice images of ventricles for PBS NS (top-left panel), PBS S (bottom-left panel), Cis NS (top-right panel), and Cis S (bottom-right panel), showing enlarged ventricle in Cis NS animal.

FIG. 6B is a plot illustrating that Cis NS animals had significantly larger ventricles while Cis S animals had comparable size of ventricles compared to PBS groups. PBS NS, n=16 slices from 8 animals; PBS S, n=14 slices from 7 animals; cisplatin NS, n=14 slices from 7 animals; cisplatin S, n=16 slices from 8 animals. 2-way ANOVA, interaction between cisplatin and GENUS F_{1,56}=8.708 **p<0.01, Tukey's multiple comparisons test, PBS NS vs. Cis NS *p<0.05, PBS S vs. Cis NS **p<0.01, PBS NS vs. Cis Sp>0.999, PBS S vs. Cis Sp=0.791.

FIG. 7A is a plot illustrating that MTX treated animals showed ˜60% survival rate 1 week after the first administration of the drug.

FIG. 7B is a plot illustrating that MTX treated animals showed impaired body weight gain PBS NS, n=8; PBS S, n=8; MTX NS, n=7; MTX S, n=7; 3-way repeated measure ANOVA, effect of MTX, F_{1, 26}=43.83, **** p<0.0001, effect of GENUS, F_{1, 26}=2.358, p=0.1367.

FIG. 7C illustrates Iba1 staining of corpus callosum for PBS NS (first panel), PBS S (second panel), MTX NS (third panel), and MTX S (fourth panel).

FIG. 7D is a plot illustrating that the Iba1 signal was significantly higher in MTX NS animals. PBS NS, n=12 slices from 6 animals; PBS S=12 slices from 6 animals; MTX NS, n=15 slices from 7 animals; MTX S, n=14 slices from 7 animals; 2-way ANOVA, interaction between MTX and GENUS F_{1, 49}=26.52, ****p<0.0001, Tukey's multiple comparisons test, PBS NS vs. MTX NS ****p<0.0001, PBS S vs. MTX NS **p<0.01, MTX NS vs. MTX S ****p<0.0001.

FIG. 7E illustrates Olig2 (red) and Hoechst (blue) staining of corpus callosum for PBS NS (first panel), PBS S (second panel), MTX NS (third panel), and MTX S (fourth panel).

FIG. 7F is a plot illustrating that GENUS increased total number of Olig2⁺ cells in the white matter of both PBS and MTX treated animals. PBS NS, n=8 slices from 4 animals; PBS S=8 slices from 4 animals; MTX NS, n=8 slices from 4 animals; MTX S, n=8 slices from 4 animals; 2-way ANOVA, effect of MTX F_{1, 28}=7.329, *p<0.05, Tukey's multiple comparisons test, MTX NS vs. MTX S *p<0.05.

FIG. 8A is a cluster plot illustrating genes that are expressed higher in microglia from Cis NS animals (positive avg_log 2FC value) and those that are expressed higher in microglia from PBS NS animals (negative avg_log 2FC value).

FIG. 8B is a cluster plot illustrating genes that are expressed higher in microglia from Cis S animals (positive avg_log 2FC value) and those that are expressed higher in microglia from Cis NS animals (negative avg_log 2FC value).

FIG. 8C is a cluster plot illustrating genes that are expressed higher in oligodendrocytes from Cis NS animals (positive avg_log 2FC value) and those that are expressed higher in oligodendrocytes from PBS NS animals (negative avg_log 2FC value).

FIG. 8D is a cluster plot illustrating genes that are expressed higher in oligodendrocytes from Cis S animals (positive avg_log 2FC value) and those that are expressed higher in oligodendrocytes from Cis NS animals (negative avg_log 2FC value).

FIG. 8E is a cluster plot illustrating genes that are expressed higher in OPCs from Cis NS animals (positive avg_log 2FC value) and those that are expressed higher in OPCs from PBS NS animals (negative avg_log 2FC value).

FIG. 8F is a cluster plot illustrating genes that are expressed higher in OPCs from Cis S animals (positive avg_log 2FC value) and those that are expressed higher in OPCs from Cis NS animals (negative avg_log 2FC value).

### DETAILED DESCRIPTION

All combinations of the foregoing concepts and additional concepts are discussed in greater detail below (provided such concepts are not mutually inconsistent) and are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

In one aspect, the present disclosure provides methods, devices, and systems for inducing gamma entrainment in one or more regions of the brain of a subject undergoing chemotherapy treatment (also sometimes referred to as just “chemotherapy”). Without being limited by theory, inducing gamma entrainment in the subject can prevent or mitigate any potential cognitive impairment that the subject may develop that is at least partly induced, caused by, a result of, and/or otherwise associated with chemotherapy treatment.

In another aspect, the present disclosure provides methods, devices, and systems for preventing, mitigating, and/or treating a cognitive impairment in a subject, where the cognitive impairment is at least partly induced, caused by, a result of, and/or otherwise associated with chemotherapy treatment of the subject. The chemotherapy treatment may be ongoing and/or have taken place prior to said preventing, mitigating, and/or treating, and may include, for example one or more of chemotherapy, hormone therapy, immunotherapy, surgery, and/or the like.

In another aspect, the present disclosure provides methods, devices, and systems for preventing, mitigating, and/or treating a cognitive impairment in a subject, where the cognitive impairment is at least partly induced, caused by, a result of, and/or otherwise associated with chemotherapy treatment of the subject. The chemotherapy treatment may be ongoing and/or have taken place prior to said preventing, mitigating, and/or treating. For example, the subject may be between rounds of chemotherapy, or have fully/partially recovered from the cancer (i.e., is a survivor).

In another aspect, the present disclosure provides methods, devices, and systems for preventing, mitigating, and/or treating inflammation in a brain region of a subject (also sometimes referred to as ‘neuroinflammation’), where the inflammation is at least partly induced, caused by, a result of, and/or otherwise associated with chemotherapy treatment of the subject. The reduction in inflammation can be due to (and as a result of the methods, devices, systems disclosed here) a reduction in the number of microglia, or astrocytes, or both, in the brain region. The brain region can include, for example, the hippocampus of the subject. Without being limited by theory, the methods, devices, and systems disclosed herein may protect one or more types of brain cells from being damaged by chemotherapy agents, and prevent inflammation from the very beginning of treatment. Additionally or alternatively, the stimulus delivered as disclosed herein may be perceived by glial cells through direct electrical signal transduction from neurons and/or through molecules secreted by other cells in response to the stimulus, leading to altered neuroinflammatory response such as decreased pro-inflammatory gene expression, cytokine release, and/or the like.

In another aspect, the present disclosure provides methods, devices, and systems for preventing, mitigating, and/or treating deoxyribonucleic acid (DNA) damage in a brain region of a subject, where the DNA damage is at least partly induced, caused by, a result of, and/or otherwise associated with chemotherapy treatment of the subject. Without being limited by theory, the stimulus delivered as disclosed herein may induce cytoprotective gene expression, thus preventing DNA damage and/or promoting it's repair.

In another aspect, the present disclosure provides methods, devices, and systems for at least partially reversing a reduction in ventricle size in a brain region of a subject, where the reduction in ventricle size is at least partly induced, caused by, a result of, and/or otherwise associated with chemotherapy treatment of the subject. Without being limited by theory, the stimulus delivered as disclosed herein may protect against cell loss or alteration in extracellular matrix, which may in turn contribute to total brain volume.

In another aspect, the present disclosure provides methods, devices, and systems for of increasing generation of at least one of oligodendrocytes or oligodendrocyte precursor cells (OPCs), or both, in a brain region of a subject. The subject may be undergoing, and/or have previously undergone, chemotherapy treatment such as, for example, treatment with a chemotherapy agent. The brain region can include white matter. Without being limited by theory, the stimulus delivered as disclosed herein may result in increased neuronal activity, which in turn promotes oligodendroglial lineage cell proliferation and increased myelination of neural axons (also sometimes referred to as “adaptive myelination”). Adaptive myelination is crucial for proper learning and memory formation, and chemo brain animal models have been shown to lack adaptive myelination.

The chemotherapy treatment can include administration, to the subject, of one or more chemotherapeutic agents. The chemotherapeutic agents can include one or more of 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abemaciclib, Abiraterone acetate, Abraxane, Acalabrutinib, Accutane, Actinomycin-D, Adcetris, Ado-Trastuzumab Emtansine, Adriamycin, Adrucil, Afatinib, Afinitor, Agrylin, Ala-Cort, Aldesleukin, Alemtuzumab, Alecensa, Alectinib, Alimta, Alitretinoin, Alkaban-AQ, Alkeran, All-transretinoic Acid, Alpelisib, Alpha Interferon, Altretamine, Alunbrig, Amethopterin, Amifostine, Aminoglutethimide, Anagrelide, Anandron, Anastrozole, Apalutamide, Arabinosylcytosine, Ara-C, Aranesp, Aredia, Arimidex, Aromasin, Arranon, Arsenic Trioxide, Arzerra, Asparaginase, Atezolizumab, Atra, Avastin, Avelumab, Axicabtagene Ciloleucel, Axitinib, Azacitidine, Balversa, Bavencio, Bcg, Beleodaq, Belinostat, Bendamustine, Bendeka, Besponsa, Bevacizumab, Bexarotene, Bexxar, Bicalutamide, Bicnu, Binimetinib, Blenoxane, Bleomycin, Blinatumomab, Blincyto, Bortezomib, Bosulif, Bosutinib, Braftovi, Brentuximab Vedotin, Brigatinib, Busulfan, Busulfex, C225, Cabazitaxel, Cablivi, Cabozantinib, Calcium Leucovorin, Calquence, Campath, Camptosar, Camptothecin-11, Capecitabine, Caplacizumab-yhdp, Caprelsa, Carac, Carboplatin, Carfilzomib, Carmustine, Carmustine Wafer, Casodex, CCI-779, Ccnu, Cddp, Ceenu, Cemiplimab-rwlc, Ceritinib, Cerubidine, Cetuximab, Chlorambucil, Cisplatin, Citrovorum Factor, Cladribine, Clofarabine, Clolar, Cobimetinib, Cometriq, Cortisone, Cosmegen, Cotellic, Cpt-11, Crizotinib, Cyclophosphamide, Cyramza, Cytadren, Cytarabine, Cytarabine Liposomal, Cytosar-U, Cytoxan, Dabrafenib, Dacarbazine, Dacogen, Dacomitinib, Dactinomycin, Daratumumab, Darbepoetin Alfa, Darolutamide, Darzalex, Dasatinib, Daunomycin, Daunorubicin, Daunorubicin Cytarabine (Liposomal), daunorubicin-hydrochloride, Daunorubicin Liposomal, DaunoXome, Daurismo, Decadron, Decitabine, Degarelix, Delta-Cortef, Deltasone, Denileukin Diftitox, Denosumab, DepoCyt, Dexamethasone, Dexamethasone Acetate, Dexamethasone Sodium Phosphate, Dexasone, Dexrazoxane, Dhad, Dic, Dinutuximab, Diodex, Docetaxel, Doxil, Doxorubicin, Doxorubicin Liposomal, Droxia, DTIC, Dtic-Dome, Duralone, Durvalumab, Eculizumab, Efudex, Ellence, Elotuzumab, Eloxatin, Elspar, Eltrombopag, Elzonris, Emapalumab-lzsg, Emcyt, Empliciti, Enasidenib, Encorafenib, Enhertu, Entrectinib, Enzalutamide, Epirubicin, Epoetin Alfa, Erbitux, Erdafitinib, Eribulin, Erivedge, Erleada, Erlotinib, Erwinia L-asparaginase, Estramustine, Ethyol, Etopophos, Etoposide, Etoposide Phosphate, Eulexin, Everolimus, Evista, Exemestane, Fam-Trastuzumab Deruxtecan-nxki, Fareston, Farydak, Faslodex, Fedratinib, Femara, Filgrastim, Firmagon, Floxuridine, Fludara, Fludarabine, Fluoroplex, Fluorouracil, Fluorouracil (cream), Fluoxymesterone, Flutamide, Folinic Acid, Folotyn, Fudr, Fulvestrant, G-Csf, Gamifant, Gazyva, Gefitinib, Gemcitabine, Gemtuzumab ozogamicin, Gemzar, Gilotrif, Gilteritinib, Glasdegib, Gleevec, Gleostine, Gliadel Wafer, Gm-Csf, Goserelin, Granix, Granulocyte—Colony Stimulating Factor, Granulocyte Macrophage Colony Stimulating Factor, Halaven, Halotestin, Herceptin, Herzuma, Hexadrol, Hexalen, Hexamethylmelamine, Hmm, Hycamtin, Hydrea, Hydrocort Acetate, Hydrocortisone, Hydrocortisone Sodium Phosphate, Hydrocortisone Sodium Succinate, Hydrocortone Phosphate, Hydroxyurea, Ibrance, Ibritumomab, Ibritumomab Tiuxetan, Ibrutinib, Iclusig, Idamycin, Idarubicin, Idelalisib, Idhifa, Ifex, IFN-alpha, Ifosfamide, IL-11, IL-2, Imbruvica, Imatinib Mesylate, Imfinzi, Imidazole Carboxamide, Imlygic, Inlyta, Inotuzumab Ozogamicin, INREBIC, Interferon-Alfa, Interferon Alfa-2b (PEG Conjugate), Interleukin-2, Interleukin-11, Intron A (interferon alfa-2b), Ipilimumab, Iressa, Irinotecan, Irinotecan (Liposomal), Isotretinoin, Istodax, Ivosidenib, Ixabepilone, Ixazomib, Ixempra, Jakafi, Jevtana, Kadcyla, Keytruda, Kidrolase, Kisqali, Kymriah, Kyprolis, Lanacort, Lanreotide, Lapatinib, Larotrectinib, Lartruvo, L-Asparaginase, Lbrance, Lcr, Lenalidomide, Lenvatinib, Lenvima, Letrozole, Leucovorin, Leukeran, Leukine, Leuprolide, Leurocristine, Leustatin, Libtayo, Liposomal Ara-C, Liquid Pred, Lomustine, Lonsurf, Lorbrena, Lorlatinib, L-PAM, L-Sarcolysin, Lumoxiti, Lupron, Lupron Depot, Lynparza, Marqibo, Matulane, Maxidex, Mechlorethamine, Mechlorethamine Hydrochloride, Medralone, Medrol, Megace, Megestrol, Megestrol Acetate, Mekinist, Mektovi, Melphalan, Mercaptopurine, Mesna, Mesnex, Methotrexate, Methotrexate Sodium, Methylprednisolone, Meticorten, Midostaurin, Mitomycin, Mitomycin-C, Mitoxantrone, Mogamulizumab KPKC, Moxetumomab, M-Prednisol, MTC, MTX, Mustargen, Mustine, Mutamycin, Mvasi, Myleran, Mylocel, Mylotarg, Navelbine, Necitumumab, Nelarabine, Neosar, Neratinib, Nerlynx, Neulasta, Neumega, Neupogen, Neulasta Onpro, Nexavar, Nilandron, Nilotinib, Nilutamide, Ninlaro, Nipent, Niraparib, Nitrogen Mustard, Nivolumab, Nolvadex, Novantrone, Nplate, Nubeqa, Obinutuzumab, Octreotide, Octreotide Acetate, Odomzo, Ofatumumab, Olaparib, Olaratumab, Omacetaxine, Oncospar, Oncovin, Onivyde, Ontak, Onxal, Opdivo, Oprelvekin, Orapred, Orasone, Osimertinib, Otrexup, Oxaliplatin, Paclitaxel, Paclitaxel Protein-bound, Palbociclib, Pamidronate, Panitumumab, Panobinostat, Panretin, Paraplatin, Pazopanib, Pediapred, Peg Interferon, Pegaspargase, Pegfilgrastim, Peg-Intron, PEG-L-asparaginase, Pembrolizumab, Pemetrexed, Pentostatin, Perj eta, Pertuzumab, Phenylalanine Mustard, Piqray, Platinol, Platinol-AQ, Pomalidomide, Pomalyst, Ponatinib, Portrazza, Poteligeo, Pralatrexate, Prednisolone, Prednisone, Prelone, Procarbazine, Procrit, Proleukin, Prolia, Prolifeprospan 20 with Carmustine Implant, Promacta, Provenge, Purinethol, Radium 223 Dichloride, Raloxifene, Ramucirumab, Rasuvo, Regorafenib, Revlimid, Rheumatrex, Ribociclib, Rituxan, Rituxan Hycela, Rituximab, Rituximab Hyalurodinase, Roferon-A (Interferon Alfa-2a), Romidepsin, Romiplostim, Rozlytrek, Rubex, Rubidomycin Hydrochloride, Rubraca, Rucaparib, Ruxolitinib, Rydapt, Sandostatin, Sandostatin LAR, Sargramostim, Siltuximab, Sipuleucel-T, Soliris, Solu-Cortef, Solu-Medrol, Somatuline, Sonidegib, Sorafenib, Sprycel, Sti-571, Stivarga, Streptozocin, SU11248, Sunitinib, Sutent, Sylvant, Synribo, Tafinlar, Tagraxofusp-erzs, Tagrisso, Talimogene Laherparepvec, Talazoparib, Talzenna, Tamoxifen, Tarceva, Targretin, Tasigna, Taxol, Taxotere, Tecentriq, Temodar, Temozolomide, Temsirolimus, Teniposide, Tespa, Thalidomide, Thalomid, TheraCys, Thioguanine, Thioguanine Tabloid, Thiophosphoamide, Thioplex, Thiotepa, Tibsovo, Tice, Tisagenlecleucel, Toposar, Topotecan, Toremifene, Torisel, Tositumomab, Trabectedin, Trametinib, Trastuzumab, Treanda, Trelstar, Tretinoin, Trexall, Trifluridine/Tipiricil, Triptorelin pamoate, Trisenox, Truxima, Tspa, T-VEC, Tykerb, Unituxin, Valrubicin, Valstar, Vandetanib, VCR, Vectibix, Velban, Velcade, Vemurafenib, Venclexta, Venetoclax, VePesid, Verzenio, Vesanoid, Viadur, Vidaza, Vinblastine, Vinblastine Sulfate, Vincasar Pfs, Vincristine, Vincristine Liposomal, Vinorelbine, Vinorelbine Tartrate, Vismodegib, Vitrakvi, Vizimpro, Vlb, VM-26, Vorinostat, Votrient, VP-16, Vumon, Vyxeos, Xalkori Capsules, Xeloda, Xgeva, Xofigo, Xospata, Xtandi, Yervoy, Yescarta, Yondelis, Zaltrap, Zanosar, Zarxio, Zejula, Zelboraf, Zevalin, Zinecard, Ziv-aflibercept, Zoladex, Zoledronic Acid, Zolinza, Zometa, Zydelig, Zykadia, or Zytiga.

The chemotherapy treatment can be for purposes of mitigating and/or treating, in the subject, one or more of a carcinoma, a sarcoma, a melanoma, a lymphoma, and/or a leukemia. Non-limiting examples of cancer that the subject will/is/was undergoing chemotherapy treatment can include Acute Lymphoblastic Leukemia (ALL), Acute Myeloid Leukemia (AML), Cancer in Adolescents, Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Childhood Astrocytoma, Childhood Atypical Teratoid/Rhabdoid Tumor of the Central Nervous System, Basal Cell Carcinoma of the Skin, Bile Duct Cancer, Bladder Cancer, Bone Cancer, Ewing Sarcoma, Osteosarcoma, Malignant Fibrous Histiocytoma, Brain Tumors, Breast Cancer, Bronchial Tumor (Lung Cancer), Burkitt Lymphoma, Carcinoid Tumor (Gastrointestinal), Carcinoma of Unknown Primary, Childhood Cardiac (Heart) Tumor, Childhood Atypical Teratoid/Rhabdoid Tumor, Medulloblastoma, CNS Embryonal Tumors, Childhood (Brain Cancer) Childhood Germ Cell Tumor, Primary CNS Lymphoma, Cervical Cancer, Childhood Cancers, Unusual Cancers of Childhood, Unusual, Cholangiocarcinoma, Childhood Chordoma, Chronic Lymphocytic Leukemia (CLL), Chronic Myelogenous Leukemia (CML), Chronic Myeloproliferative Neoplasm, Colorectal Cancer, Childhood Craniopharyngioma, Cutaneous T-Cell Lymphoma, Ductal Carcinoma In Situ (DCIS), Childhood Embryonal Tumors, Childhood Medulloblastoma and Other Central Nervous System Cancers, Endometrial Cancer, Childhood Ependymoma, Esophageal Cancer, Esthesioneuroblastoma (Head and Neck Cancer), Ewing Sarcoma (Bone Cancer), Childhood Extracranial Germ Cell Tumor, Extragonadal Germ Cell Tumor, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumor (GIST) (Soft Tissue Sarcoma), Childhood Central Nervous System Germ Cell Tumor, Childhood Extracranial Germ Cell Tumor, Extragonadal Germ Cell Tumor, Ovarian Germ Cell Tumor, Testicular Cancer, Gestational Trophoblastic Disease, Hairy Cell Leukemia, Head and Neck Cancer, Childhood Heart Tumors, Hepatocellular (Liver) Cancer, Langerhans Cell Histiocytosis, Hodgkin Lymphoma, Hypopharyngeal Cancer, Intraocular Melanoma, Islet Cell Tumor, Pancreatic Neuroendocrine Tumor, Kaposi Sarcoma (Soft Tissue Sarcoma), Kidney (Renal Cell) Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and/or Tracheobronchial Tumor), Lymphoma, Male Breast Cancer, Malignant Fibrous Histiocytoma of Bone and Osteosarcoma, Melanoma, Intraocular Melanoma (Eye), Merkel Cell Carcinoma, Malignant Mesothelioma, Metastatic Cancer, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasm, Mycosis Fungoides, Myelodysplastic Syndrome, Myelodysplastic/Myeloproliferative Neoplasm, Chronic Myelogenous Leukemia, Acute Myeloid Leukemia, Chronic Myeloproliferative Neoplasm, Nasal Cavity and Paranasal Sinus Cancer, Nasopharyn4geal Cancer, Neuroblastoma, Non-Hodgkin Lymphoma, Non-Small Cell Lung Cancer, Oral Cancer, Lip and Oral Cavity Cancer, Oropharyngeal Cancer, Osteosarcoma and Malignant Fibrous Histiocytoma of Bone, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer, Parathyroid Cancer, Penile Cancer, Pharyngeal Cancer, Pheochromocytoma Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Childhood Rhabdomyosarcoma, Salivary Gland Cancer, Childhood Rhabdomyosarcoma, Childhood Vascular Tumors, Ewing Sarcoma, Kaposi Sarcoma, Osteosarcoma, Soft Tissue Sarcoma, Uterine Sarcoma, Sézary Syndrome, Skin Cancer, Small Cell Lung Cancer, Small Intestine Cancer, Soft Tissue Sarcoma, quamous Cell Carcinoma of the Skin, Metastatic Squamous Neck Cancer with Occult Primary, Stomach (Gastric) Cancer, Cutaneous T-Cell Lymphoma, Testicular Cancer, Throat Cancer, Thymoma, Thymic Carcinoma, Thyroid Cancer, Tracheobronchial Tumor, Transitional Cell Cancer of the Renal Pelvis and Ureter, Carcinoma of Unknown Primary, Unusual Cancers of Childhood, Transitional Cell Cancer of Ureter and Renal Pelvis, Urethral Cancer, Endometrial Uterine Cancer, Uterine Sarcoma, Vaginal Cancer, Vascular Tumor, Vulvar Cancer, Wilms Tumor and Other Childhood Kidney Tumors, or Cancer in Young Adults.

This cancer-related/chemotherapy-related cognitive impairment can include, but is not limited to, one or more of short-term memory loss, attention deficit, increased anxiety, and decreased problem-solving ability. The methods, devices, and systems described herein, including for the treatment of the cognitive impairment, can include delivering a stimulus to the subject to induce gamma entrainment in one or more regions of the brain of the subject. The stimulus can include an auditory stimulus, a visual stimulus, or both. In some aspects, the stimulus includes both an auditory stimulus and a visual stimulus. In some aspects, methods, devices, and systems for delivering the auditory stimulus and/or the visual stimulus can be similar to that described in Intl. Application No. PCT/US2016/063536, and/or in Intl. Application No. PCT/US2018/051785, and/or in Intl. Application No. PCT/US2018/055258, the disclosure of each of which is incorporated by reference in its entirety. The auditory stimulus and/or the visual stimulus can independently be non-invasive, or invasive, or a combination thereof.

The stimulus can be administered invasively and/or non-invasively. The term “non-invasive,” as used herein, refers to methods, devices, and systems which do not require surgical intervention or manipulations of the body, such as injection or implantation of a composition or a device. The term “invasive,” as used herein, refers to methods, devices, and systems which do require surgical intervention or manipulations of the body. Non-limiting examples of non-invasive administration of stimulus can include audio, visual (e.g., flickering lights), haptic stimulation, and/or the like. Non-limiting examples of invasive administration of stimulus can include visual, audio, and/or haptic stimulations combined with an injection or implantation of a composition (e.g., a light-sensitive protein) or a device (e.g., an integrated fiber optic and solid-state light source). Other examples of invasive administration can include magnetic and/or electrical stimulation via an implantable device or a device disposed on the body of the subject.

The combined stimulus may include any purposive, detectable change in the internal (e.g., when the combined stimulus is administered invasively) or external (e.g., when the combined stimulus is administered non-invasively) environment of the subject that directly or ultimately induces gamma oscillations/results in gamma entrainment in at least one brain region. For example, the combined stimulus may be designed to at least stimulate electromagnetic radiation receptors (e.g., photoreceptors, infrared receptors, and/or ultraviolet receptors) and sound receptors, and may further stimulate one or more of mechanoreceptors (e.g., mechanical stress and/or strain), nociceptors (i.e., pain), electroreceptors (e.g., electric fields), magnetoreceptors (e.g., magnetic fields), hydroreceptors, chemoreceptors, thermoreceptors, osmoreceptors, or proprioceptors (i.e., sense of position). The absolute threshold or the minimum amount of sensation needed to elicit a response from such receptors may vary based on the type of stimulus and the subject. In some embodiments, the visual and/or auditory stimulus is adapted based on individual sensitivity to either or both stimuli.

The auditory stimulus can have a frequency of less than about 20 Hz, about 20 Hz, about 30 Hz, about 40 Hz, about 50 Hz, about 60 Hz, or more than 60 Hz, including all values and sub-ranges in between. As an example, the auditory stimulus can include a click train/clicking sound with a click frequency of about 35 clicks/s to about 45 clicks/s. In some aspects, the click frequency can be about 40 Hz. The duty cycle of the auditory stimulus can be about 4%, about 10%, about 20%, about 50%, about 60%, about 80%, including all values and sub-ranges in between.

The visual stimulus can have a frequency of less than about 20 Hz, about 20 Hz, about 30 Hz, about 40 Hz, about 50 Hz, about 60 Hz, or more than 60 Hz, including all values and sub-ranges in between. As an example, the visual stimulus can include a flashing/flickering light with a flicker frequency of about 35 Hz to about 45 Hz. In some aspects, the flicker frequency can be about 40 Hz. The duty cycle of the flashing light can be about 4%, about 10%, about 20%, about 50%, about 60%, about 80%, including all values and sub-ranges in between.

Without being limited by theory, cognitive function critically depends on the precise timing of oscillations in neural network activity, specifically in the gamma frequency (e.g., about 20 Hz to about 100 Hz, about 20 Hz to about 80 Hz, or about 20 Hz to about 60 Hz), a rhythm that is linked to attention and working memory. Because these oscillations emerge from synaptic activity, they can provide a direct link between the molecular properties of neurons and higher level, coherent brain activity.

The combined stimulus can be administered immediately after (i.e., with no waiting period) the chemotherapy treatment (e.g., after a single cisplatin administration), or alternatively about an hour, at least an hour, 2 hours, 4 hours, 6 hours, 12 hours, 24 hours or more, after the chemotherapy treatment, including all values and sub-ranges in between.

The combined stimulus can be administered after chemotherapy treatment has been completed (e.g., after a last cisplatin administration) such as, for example, an hour, a day, 2 days, a week, a month, 2 months, 6 months, 9 months, a year, more than a year after the completion of chemotherapy treatment, including all values and sub-ranges in between. The administration after chemotherapy treatment can be periodic such as, for example, every hour, 2 hours, 4 hours, 6 hours, 12 hours, 24 hours or more, including all values and sub-ranges in between.

Each administration of the combined stimulus can be for a duration of about 15 minutes, about 30 minutes, about an hour, about two hours, about four hours more than four hours, including all values and sub-ranges in between.

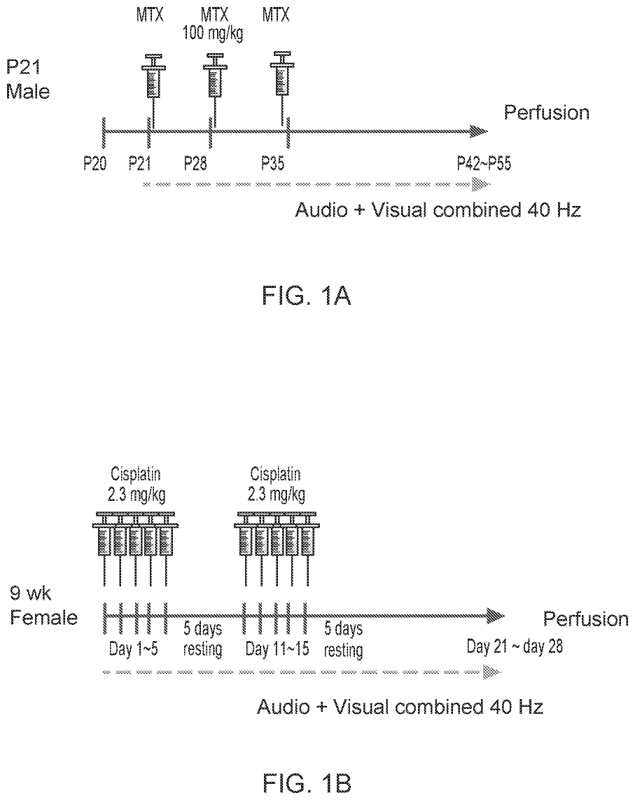

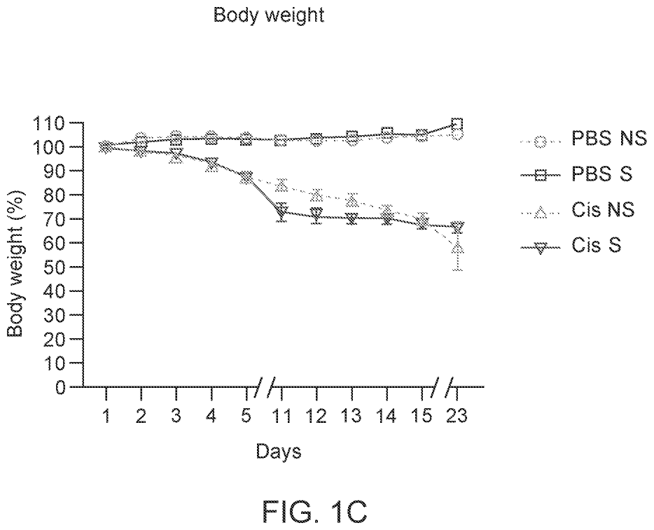

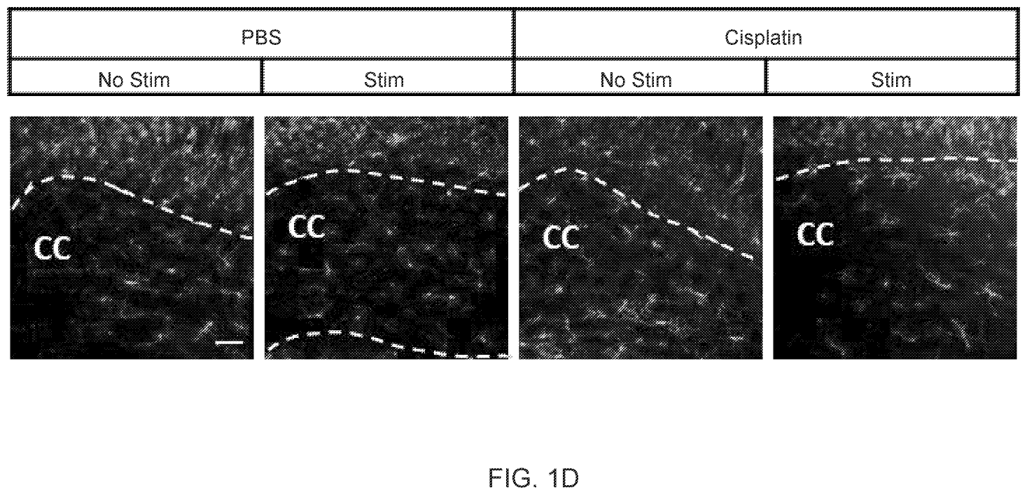

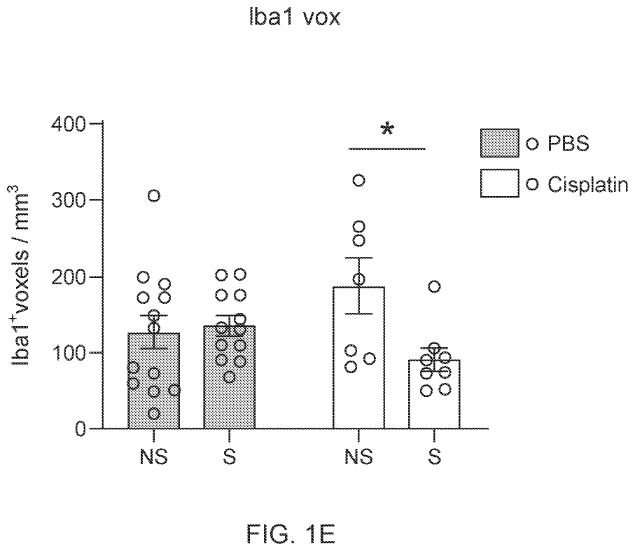

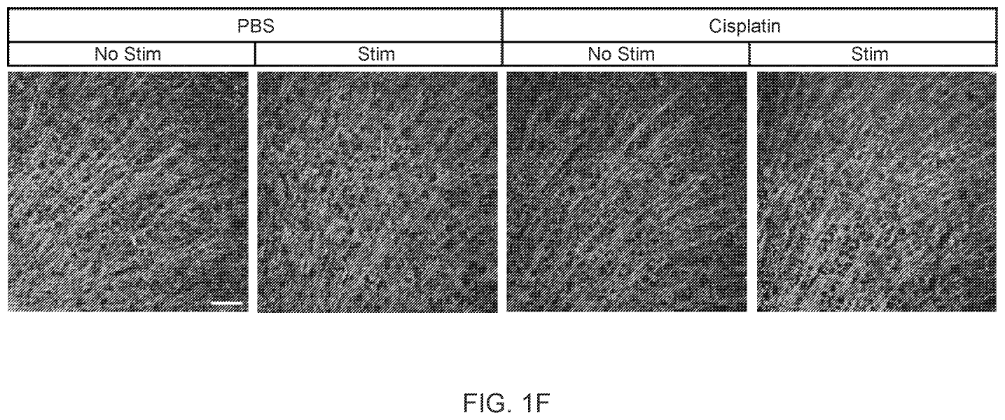

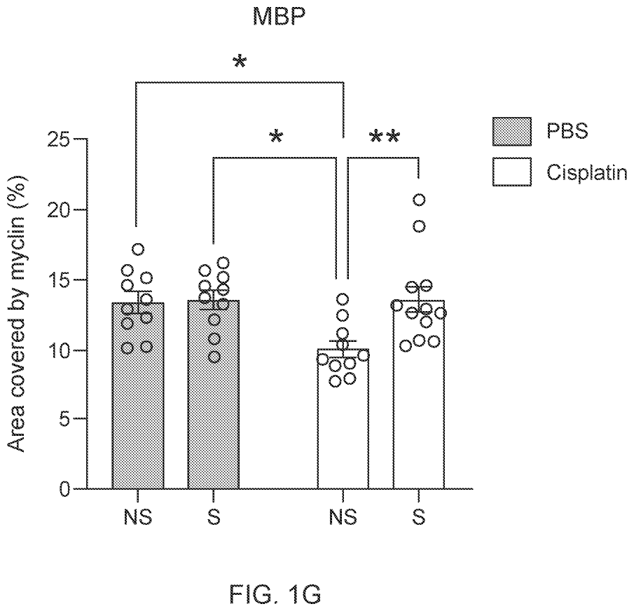

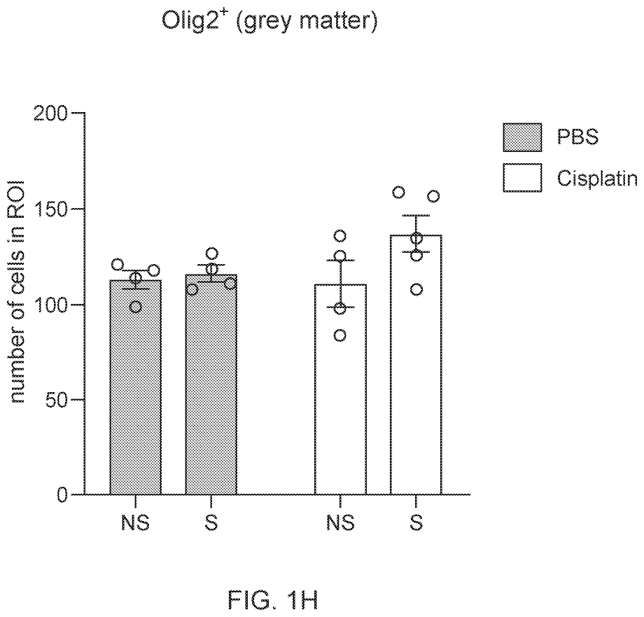

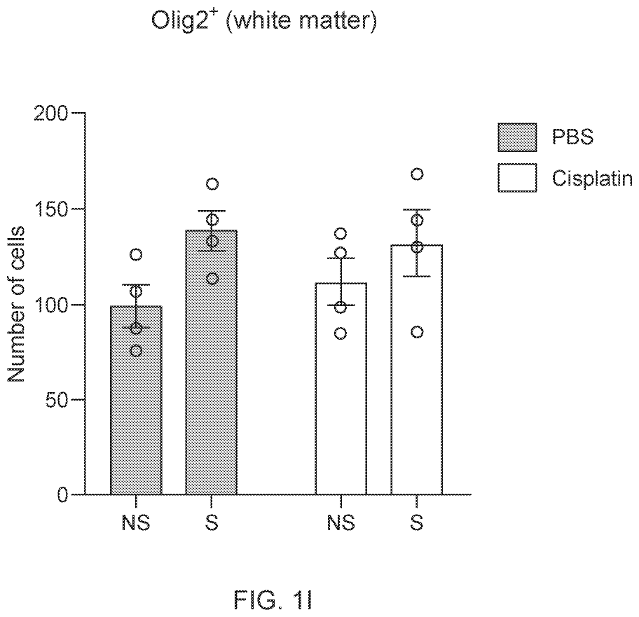

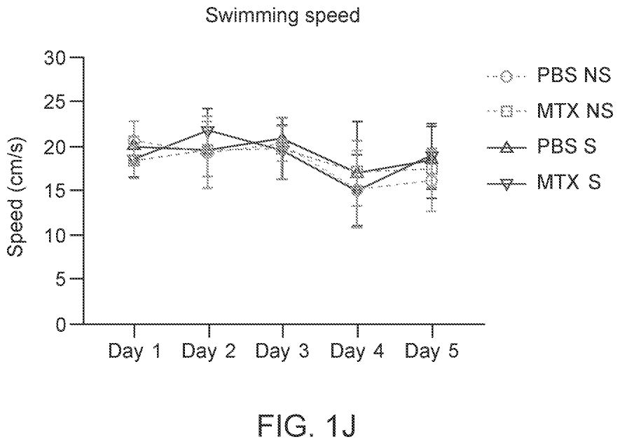

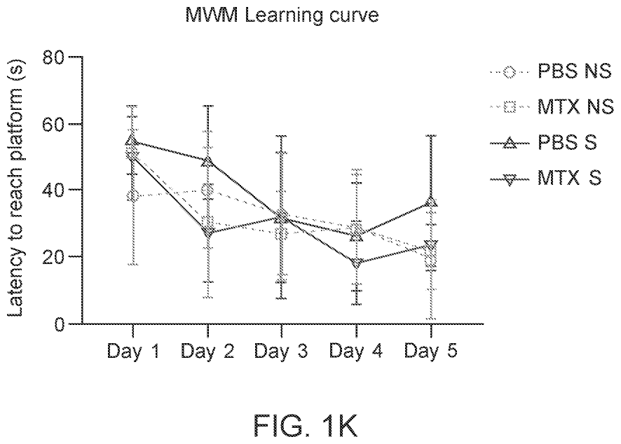

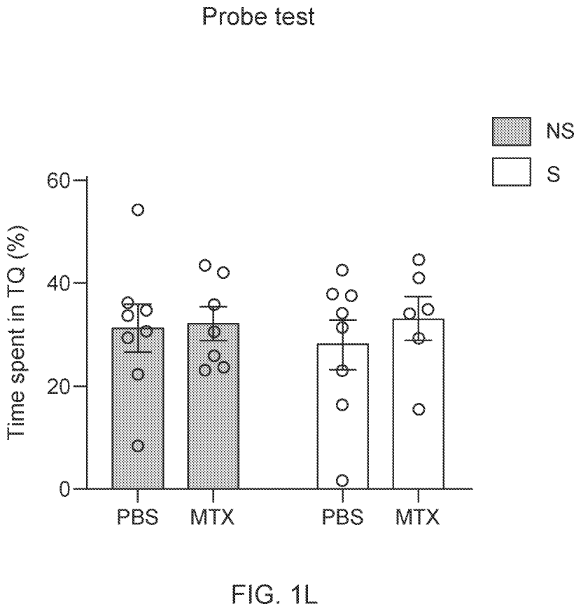

In some cases, the combined stimulus can be administered as illustrated in FIG. 1A and/or FIG. 1B (depending on the chemotherapy agent), as explained in greater detail in Example 1.

Without being limited by theory, administration of the combined stimulus may reduce a rate of increase of demyelination in the brain of the subject, and/or reverse demyelination in the brain of the subject. Additionally or alternatively, administration of the combined stimulus may reduce a rate of increase of microglial filtration into one or more regions of white matter in the brain of the subject, and/or reverse microglial filtration into one or more regions of white matter in the brain of the subject.

### Systems and Devices

Systems and devices for delivering the combined stimulus as disclosed herein can generally include any suitable stimulus emitting and/or delivery device. Examples of such devices for generating and/or delivering a visual stimulus can include, but are not limited to, flash lamps, pulsed lasers, light emitting diodes including laser diodes (and generally, any solid-state light source), intense pulsed light (IPL) sources, a device screen (e.g., the screen of a Smartphone, a laptop, a desktop computer, and/or the like), combinations thereof, and/or the like. Examples of such devices for generating and/or delivering an audio stimulus can include, but are not limited to, electroacoustic transducers, speakers, headphones, and/or the like. Examples of such devices for generating and/or delivering a haptic stimulus can include, but are not limited to, actuators (including eccentric rotating mass actuators, linear resonant actuators, magnetic voice coils, piezoelectric actuators, and/or the like), motors, focused ultrasound, and/or the like.

In some embodiments, the visual stimulus and the auditory stimulus are synchronized/in phase. In some embodiments, the visual stimulus and the auditory stimulus are out of phase by from −180 to 0 degrees or from 0 to 180 degrees, including all values and sub-ranges in between. As used herein, “phase” refers to lag between the auditory stimulus and the visual stimulus expressed in degrees, where 0 degrees means simultaneous/in-phase/synchronous auditory and visual stimulus and −180 or +180 refers to alternating visual and auditory stimulus.

By way of example, in some embodiments, the visual stimulus can include repeated 12.5 ms light on then 12.5 ms light off. As another example, the light emitting device can include a light-emitting diode with 40-80 W power. As another example, the auditory stimulus can include a 10 kHz tone played at 40 Hz with a duty cycle of about 4% to about 80%. As yet another example, the visual stimulus can include a light flickered at 40 Hz for 10 s period with a duty cycle of about 10% to about 80%.

In some cases, systems and devices for delivering the combined stimulus can also generally include a processor and a memory/database. All components of the systems and devices can be in communication with each other, including with the stimulus-emitting/delivery device. It will also be understood that the database and the memory can be separate data stores. In some embodiments, the memory/database can constitute one or more databases. Further, in other embodiments, at least one database can be external to the system/device. The system/device can also include one or more input/output (I/O) interfaces (not shown), implemented in software and/or hardware, for other components of the system/device, and/or external to the system/device, to interact with the system/device.

The memory/database can encompass, for example, a random access memory (RAM), a memory buffer, a hard drive, a database, an erasable programmable read-only memory (EPROM), an electrically erasable read-only memory (EEPROM), a read-only memory (ROM), Flash memory, and/or so forth. The memory/database can store instructions to cause the processor to execute processes and/or functions associated with the system/device. For example, the memory/database can store stimulus parameters (e.g., frequency, amplitude, duty cycle, etc.), processor executable instructions to control the stimulus-emitting device to emit the stimulus according to the stimulus parameters, and/or the like.

The processor can be any suitable processing device configured to run and/or execute a set of instructions or code associated with the system/device. The processor can be, for example, a general purpose processor, a Field Programmable Gate Array (FPGA), an Application Specific Integrated Circuit (ASIC), a Digital Signal Processor (DSP), and/or the like.

### Example—Gamma Entrainment Using Sensory Stimulus (GENUS) Rescues Cognitive Impairment in Chemo Brain Animal Models

Cancer patients often suffer from a neurological condition called chemotherapy induced cognitive impairment, or chemo brain, which may last for the rest of their life. Despite the increasing number of chemo brain patients, neither the mechanism nor cure for the symptom is well studied. Here, it was tested if Gamma Entrainment Using Sensory stimuli (GENUS) can be utilized as a tool to treat chemo brain. It is shown that GENUS alleviates cisplatin-induced symptoms such as demyelination, neuroinflammation, neurodegeneration, impaired neurogenesis and synaptic loss. These alterations by GENUS eventually lead to significant improvements of cognitive function in the mouse model. Furthermore, it is shown that the effect of GENUS was not limited to cisplatin-induced chemo brain, but also applies to methotrexate (MTX)-induced chemo brain mouse model, demonstrating that GENUS can be a versatile treatment for a wide range of chemo brain patients treated with diverse chemotherapy agents.

MTX Induced Chemo Brain Model

P21 C57/BL6J male mouse received i.p. injection of either 100 mg/kg MTX or volume-matched PBS on P21, P28, P35 (i.e., 21 days after birth/21 days old, 28 days old, 35 days old) (FIG. 1A). Stim (S) group received 1 hr of 40 Hz audio+visual sensory stimulation immediately after the first injection, and then received 1 hr stimulation every day after that day's injection until the day before they were sacrificed. No stim (NS) group stayed under a dim light for 1 hr immediately after the first injection, and then stayed under a dim light for 1 hr every day until the day before they were sacrificed. Every animal was single-caged in a caged covered with a black-plastic bag on every wall but one side for the duration of 1 hr stim/sham period and then group housed with their littermates. All animals were handled and sacrificed as approved by the Massachusetts Institute of Technology Committee on Animal Care (MIT CAC).

Cisplatin Induced Chemo Brain Model

9 weeks old C57/BL6J female mouse received i.p. injection of either 2.3 mg/kg cisplatin or volume-matched PBS for 5 consecutive days, had 5 days resting period and then received another 5 days of 2.3 mg/kg cisplatin or volume-matched PBS (FIG. 1B). Stim (S) and no stim (NS) went through the same procedure as described in MTX treated animals. All animals were handled and sacrificed as approved by the MIT CAC.

EdU Labeling

40 mg/kg EdU (Thermo Fisher, A10044) was injected for 3 consecutive days according to experimental schedule described in results section. Click-iT EdU Alexa Fluor 647 imaging kit (Thermo Fisher, C10340) was used to stain brain sections according to manufacturer's protocol.

Open Field Test (OFT)

Animals were placed in the center of 45×45 cm×40 cm (W×L×H) white acrylic box and their movement were tracked with a behavior tracking software (EthoVision XT, nodulus) for 10 min. 22.5 cm×22.5 cm area in the center of the box was marked as ‘center area’.

Puzzle Box Test

Animals were placed in a testing arena containing a lighted (55 cm×28 cm) and a dark (15 cm×28 cm) compartment with a connecting tunnel (4 cm×4 cm×2.5 cm), facing the farthest wall from the dark compartment. 3 trials were performed for 3 days. 1 introductory period without any obstacles blocking the tunnel and 2 ‘easy task’ in which animals have to pass a corridor (4 cm×4 cm×12 cm). On day 2, 1 ‘easy task’ and 2 ‘intermediate task’ in which the corridor is blocked with sawdust and the animals have to dig through to reach the dark chamber were performed. On day 3, 1 ‘intermediate task’ and 2 ‘hard task’ where the tunnel was plugged with a tissue were performed. Time of the first entry to the dark chamber was recorded for every trials. If an animal failed to reach the dark chamber within 5 min, the animal was removed from the apparatus and time to reach the dark chamber was marked as 5 min.

Morris Water Mazw (MWM) Test

A circular cylinder with 122 cm diameter was filled with tap water (22° C.-24° C.) and a white paint was added to make the water opaque. The area was divided into 4 equal quardrants and a 10 cm diameter platform was placed in the center of the target quardrant (TQ), the platform being 1 inch lower than the water level. On the first day of training, animals were placed on the platform for 30 seconds, and then retrieved from the water (priming). After 1 minute of priming, animals were placed into the chamber facing the chamber wall. Time to reach the platform was measured when the animal was sitting on the platform for >1s. For 5 consecutive days, animals performed two trials, spaced 1 min. After 5 days of training, the platform was removed from the chamber, and the animals were placed in the center of the chamber. For 1 min duration, time spent in TQ were measured Novel Object Recognition (NOR)

Animals were habituated in an OFT box for 10 min, 3 consecutive days. On the 4^{th} day, two identical wooden blocks (Premium wooden building blocks set, Cubbie Lee) were placed in the chamber, and animals were allowed to explore the objects for 5 min, then the animals were returned to their home cage for 5 min. In the test phase, one of the wooden block is switched to a novel wooden block with a different shape, and the time spent exploring the familiar and new object was measured for 10 min. Discrimination ratio was calculated as time spent to explore the new object divided by the sum of time spent to explore both old and new object.

$$Discrimination ratio = \frac{Time spent to explore new object}{\begin{matrix}Time spent to explore new object + \\ Time spent to explore familiar object\end{matrix}}$$

Immunohistochemistry

30˜40 μm thick vibratome sectioned brain slices were washed in PBS, then incubated in a blocking solution (3% normal donkey serum (Millipore Sigma, S30-M) and 0.3% Triton X-100 (Sigma-Aldrich, T8787-100ML) in PBS) for 2 hrs. Then, slices were treated with a blocking solution containing α-Iba1 (1:500, Abcam, ab178846), α-GFAP (1:1000, Novus Biologicals, NBP1-05198), α-MBP (1:500, Millipore, AB9348), α-yH2AX (1:500, EMD Millipore, 05-636), α-Iba1 (1:500, SYSY, 234004), α-Olig2 (1:200, Abcam, ab109186), α-Pdgfra (1:100, Abcam, ab90967) and α-Dcx (1:500, Cell signaling technology, 4604S) in a 4° C.-10° C. controlled cold room for 2 overnight. Slices were washed with PBS, and then stained with α-rabbit IgG Alexa 488 conjugated (1:500, Thermo Fisher, A-21206), α-rabbit IgG Alexa 555 conjugated (1:500, Thermo Fisher, A-27039), α-mouse IgG Alexa 488 conjugated (1:500, Thermo Fisher, A-28175), α-chicken IgY Alexa 555 conjugated (1:500, Thermo Fisher A-21437) and α-chicken IgY Alexa 488 conjugated (1:500, Thermo Fisher, A-11039) for 2 hrs at room temperature. Slices were washed with PBS and nuclei were stained with Hoechst 33342 (1:500000, Thermo Fisher, H3570).

Data Acquisition and Statistical Analysis

OFT, MWM behavior was tracked with a behavior tracking software (EthoVision XT, Nodulus). NOR and puzzle box behavior was scored by manual counting. Zeiss LSM 710 and Zeiss LSM 880 confocal microscope with Zen software was used to capture fluorescence images. A 3D image analysis software (IMARIS, Bitplane) was used to score Iba1 and GFAP positive voxels, and the area covered by MBP, ventricle size and cell number counting was scored with ImageJ software. Researcher was blinded for the experimental group during data analysis.

Single Cell RNA Sequencing

Cisplatin-based chemo brain model mice and their PBS controls were induced and given 40 Hz sensory stimulation as described herein. After 21 days from the first cisplatin injection, animals were anesthetized and euthanized with cardiac perfusion of cold DPBS. Two hippocampi from each animals were collected, and 4 hippocampi were pooled together as one sample. Tissue dissociation was performed with Adult Brain Dissociation Kit, mouse and rat (Miltenyl Biotec, 130-107-677) following the manufacturer's protocol. cDNA library for single cell sequencing was prepared with Chromium Next GEM Single Cell 3′ Kit v3.1 (10× Genomics, 1000268, 100127) following manufacturer's protocol. Library quality control and sequencing was performed at Koch Institute's Robert A. Swanson (1969) Biotechnology Center for technical support, specifically at the BioMicroCenter core.

For the analysis of sequenced data, filtered gene counts output from CellRanger was used to ensure high quality cells were present. Next, quality control was performed using Seurat v4 (10.1101/2020.10.12.335331v1) by restricting total RNA counts per unique molecular identifier (UMI) between 500 and 25000, and by limiting number of genes expressed per barcode to between 200 and 6000. UMIs were then filtered by keeping both mitochondrial and ribosomal gene percentages below 30 percent per barcode, and were filtered further by keeping the percentage of gene counts per barcode corresponding to the top 50 genes in total below 80 percent. Further, cell cycle phase weas identified and scored (10.1111/j.1365-2184.1979.tb00145.x), and cells with extreme G2M or S scores above 0.25 were removed. Next, gene counts were scaled and normalized using SCTransform (10.1186/s13059-019-1874-1). In order to cluster UMIs across biosamples, these biosamples were integrated via canonical correlation analysis (FindIntegrationAnchors/IntegrateData in Seurat) and principal component analysis was performed. Then, clustering was performed using a shared nearest neighbor approach implemented in Seurat. Next, clusters were manually assigned to cell types by using marker genes (10.1126/science.aaa1934). This manual annotation involved looking at the average expression and the percentage of UMIs in that cluster expressing that gene and determining how predictive these marker genes were of the cell type.

Differentially expressed genes were identified in a per-cluster manner. For each cluster, differentially expressed genes were identified via a Wilcoxon rank sum test for several cases, both simple: (1) cisplatin versus PBS (2) stimulated versus non-stimulated, as well as selected within conditions: (3) cisplatin versus PBS within stimulated mice (4) cisplatin versus PBS within non-stimulated mice (5) stimulated versus non-stimulated within cisplatin mice (6) stimulated versus non-stimulated within PBS mice. The within-class comparisons allow for visual comparisons between the different within-classes when compared to the comparison classes, as well as demonstrating the effect of filtering compared to the simpler cases.

For the final set of differential expressed genes, a combinatorial model was formulated using a negative binomial mixed model (10.1101/2020.09.24.311662) with a design matrix corresponding to the R formula ‘—group*treatment’, specifying combinatorial interactions between group (cisplatin versus PBS) and treatment (stimulated versus non-stimulated). For this model, multiple hypothesis correction was not implemented; FDR-based correction was performed after model application to correct for multiple comparisons.

From these differentially expressed genes, gene set analysis was performed using g:profiler (10.12688/f1000research.24956.2) on differentially expressed gene sets both below p=0.05 as well as differentially expressed gene sets below p=0.01.

The Chemo Brain Mouse Model

During the cisplatin administration period, general status of health as survival rate and body weight was observed following drug administration. While none of the animals in the cohort died, cisplatin treated animals showed significant body weight loss through the experiment, and there was no significant effect of GENUS on the body weight in either PBS or cisplatin treated group (FIG. 1C).

GENUS Restored Iba1 Composition in Corpus Callosum of Chemo Brain Mouse Model

Reactive microglial infiltration into white matter has been reported to be closely related to demyelination. Moreover, a recent study has shown that microglia triggers chemotherapy-mediated shift in brain microenvironment, which leads to the development of chemo brain. It was investigated if the microglia are one of the cell-types responsible in demyelination in chemo brain model animal brain and its reversal by GENUS. A microglial marker protein, Iba1, was stained and Iba1⁺ voxels in corpus callosum were counted with IMARIS 3D image analysis software (FIGS. 1D, 1E). It was found cisplatin treatment without GENUS (Cis NS) animals had higher count of Iba1⁺ voxels in corpus callosum compared to other groups and GENUS was able to reverse the Iba1⁺ voxel count to a comparable level to PBS control groups (FIG. 1E).

GENUS Treatment Protected the Brain from Cisplatin-Induced Demyelination

Demyelination of neural axons is one of the most commonly reported hallmark of chemo brain in both human cancer patients and animal models. Therefore, it was first tested if GENUS was able to protect the brain from losing myelin sheets (FIGS. 1F, 1G). In line with previous literatures, Cis NS significantly lowered the coverage of myelin basic protein (MBP) in anterior cingulate cortex (ACC) compared to PBS treated animals (FIG. 1G). However, it was found that cisplatin treated animals with GENUS (Cis S) had comparable coverage of myelin with PBS treated group (FIG. 1G).

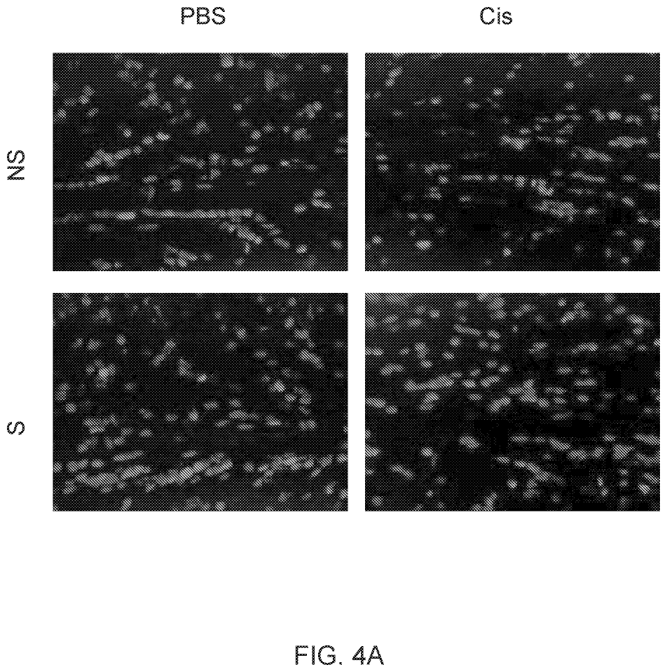

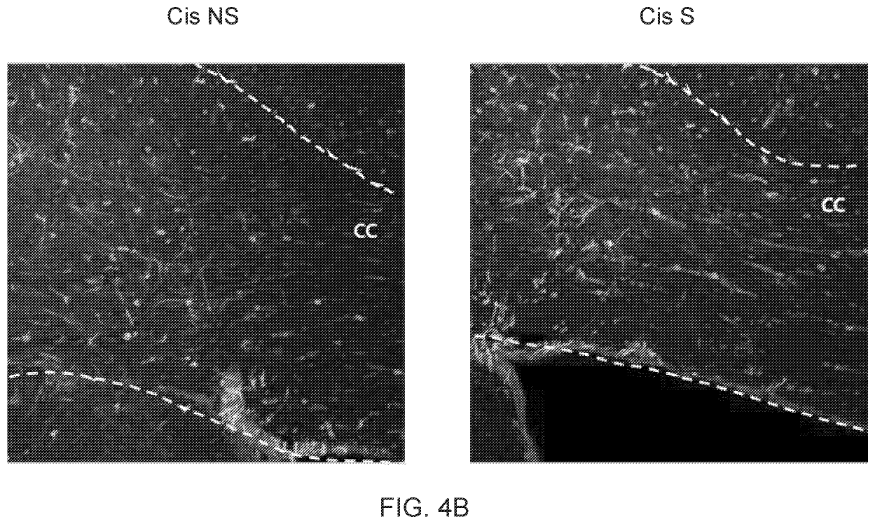

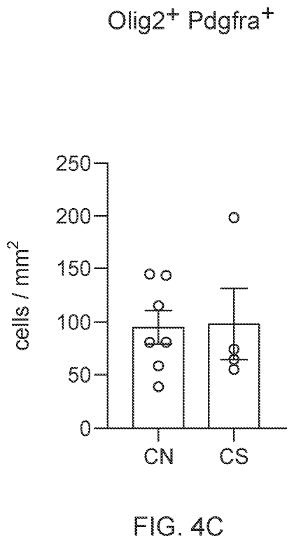

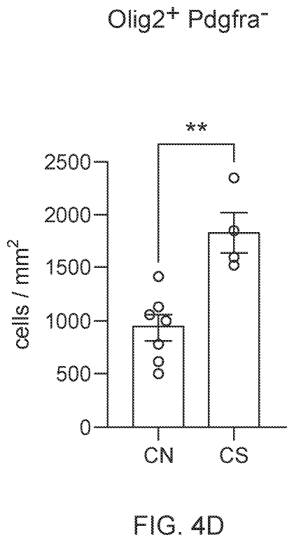

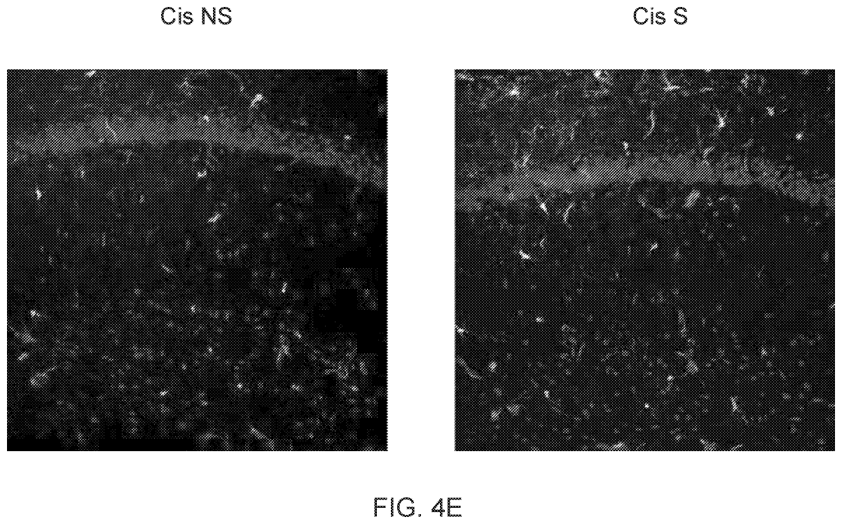

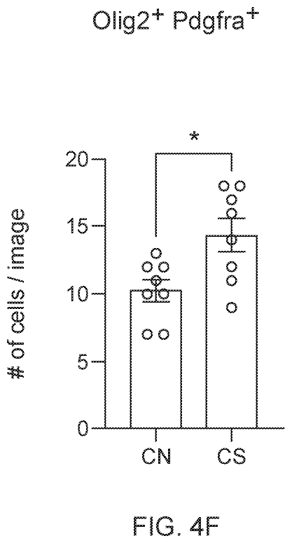

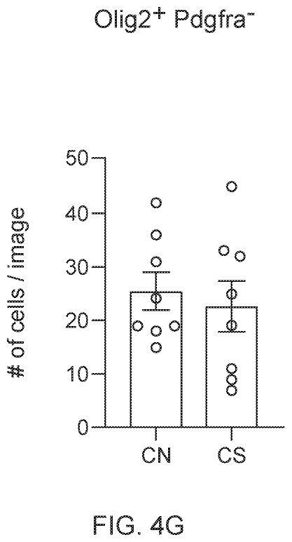

Oligodendrocyte precursor cells (OPCs) and oligodendrocytes have very low tolerance to chemotherapeutic agents that are even lower than cancer cells. The number of OPCs and oligodendrocytes (Olig2⁺ cells) were measured to see if the demyelination in Cis NS animals was the result of OPC and oligodendrocyte cell death as a toxic effect of cisplatin treatment. In grey matter (ACC), all four groups had comparable number of Olig2⁺ cells (FIG. 1H), while in white matter, GENUS treatment increased number of Olig2⁺ cells regardless of PBS or cisplatin treatment (FIGS. 1I, 4A). Long-lasting arrest of OPC differentiation was reported as a cause of demyelination in a different chemo brain animal model. Further analysis was performed of oligodendroglial cell population with Pdgfrα and Olig2 co-staining was performed to see if the ratio of OPC (Olig2⁺Pdgfrα⁺) to oligodendrocyte (Olig2⁺Pdgfrα⁻) population has changed (FIGS. 4B-4G). In chemo brain animals, GENUS had no effect on the number of OPC in corpus callosum, and the increase of Olig2⁺ cell population after GENUS was majorly contributed by the increased number of oligodendrocyte cell population (FIGS. 4B-4D). On contrary, it was found that the number of OPC has significantly increased by GENUS treatment in the hippocampus, while oligodendrocyte cell number remained comparable (FIGS. 4E-4G).

GENUS Ameliorated Cisplatin-Induced DNA Damage and Restored Neurogenesis

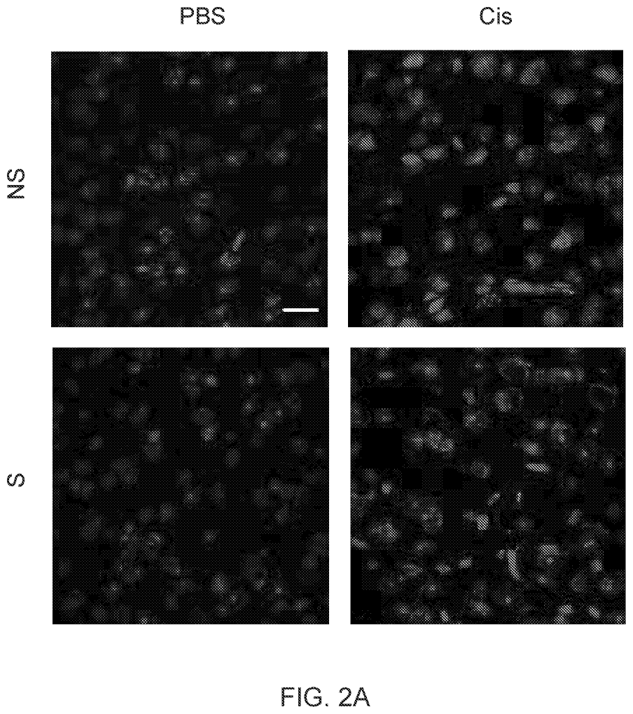

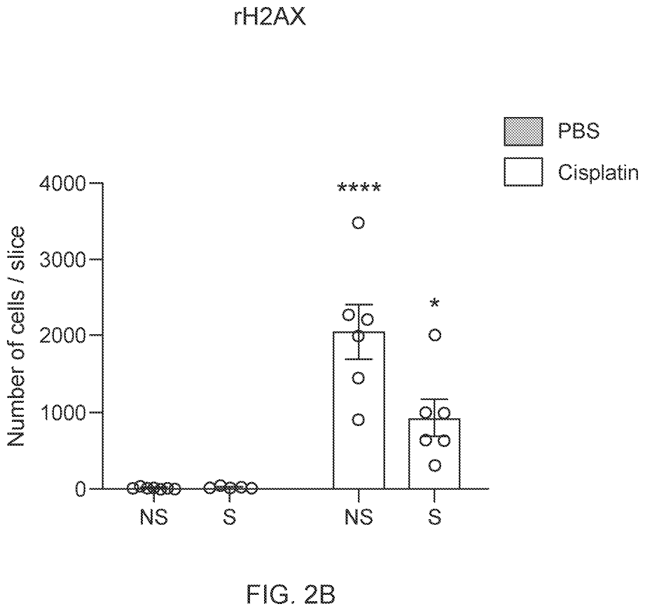

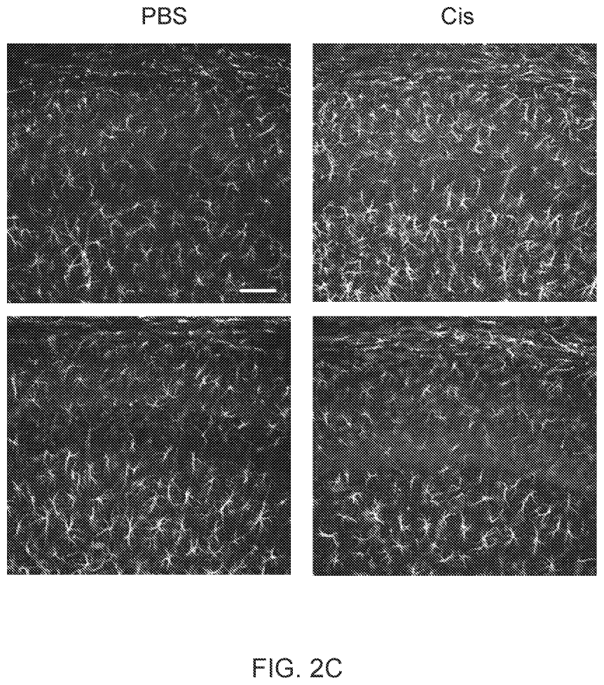

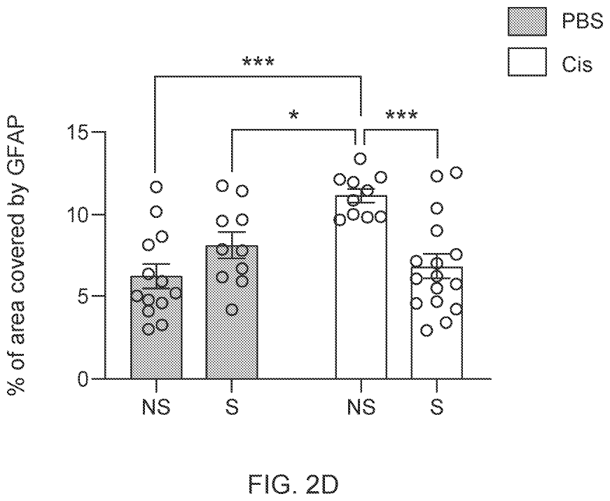

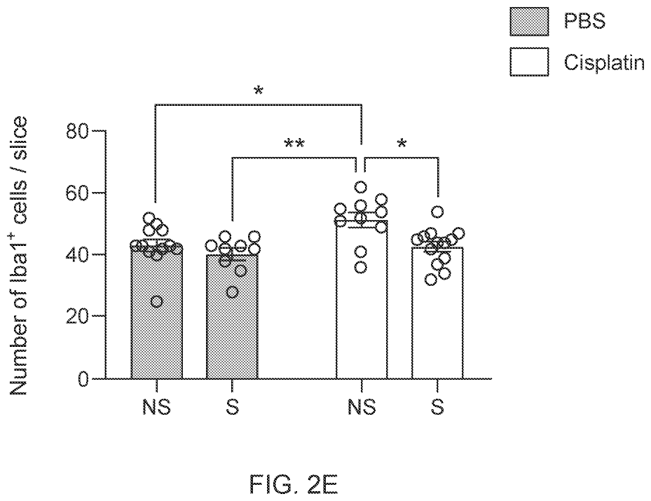

Direct damage on brain cells by the chemotherapeutic agent is one of a major suspects of why cancer patients suffer from chemo brain. As GENUS has been proved to have a neuroprotective effect and reduce DNA damage in Alzheimer's disease mouse model, it was tested if GENUS would also reduce cisplatin-induced DNA damage (FIGS. 2A, 2B). As expected, cisplatin treatment increased γH2AX signal, an indicator of DNA double strand break (DSB) damage. However, it was found that Cis NS animals had lower reduced yH2AX signature, once again proving the protective effect of GENUS against DNA damage (FIG. 2B).

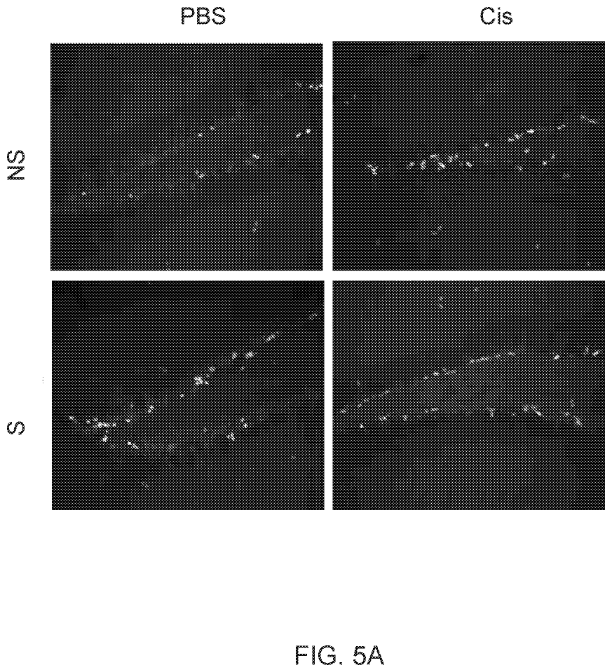

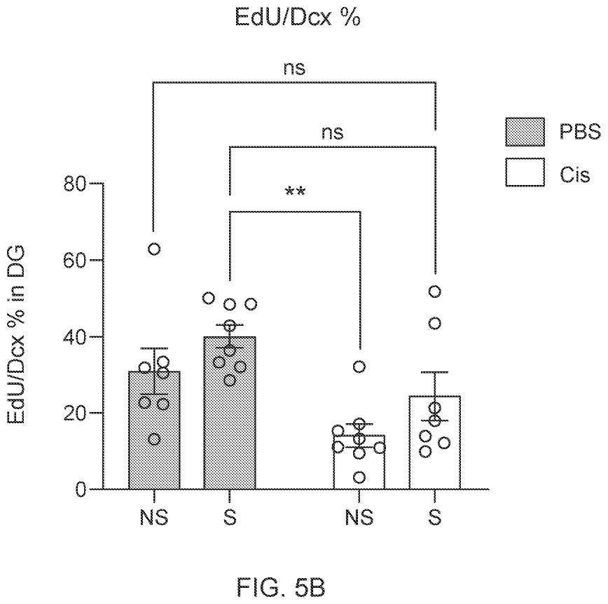

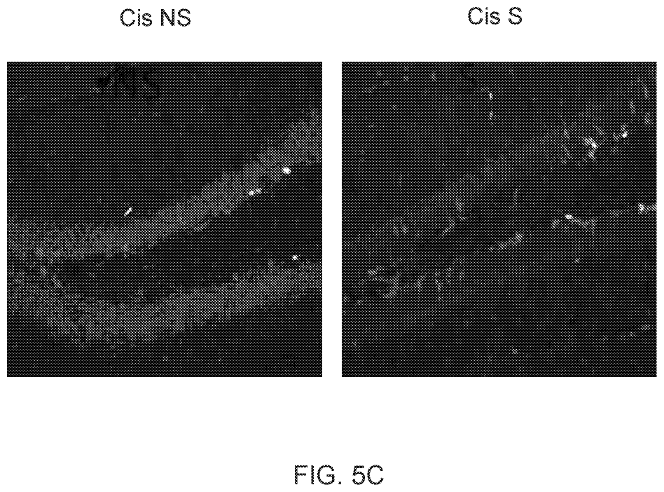

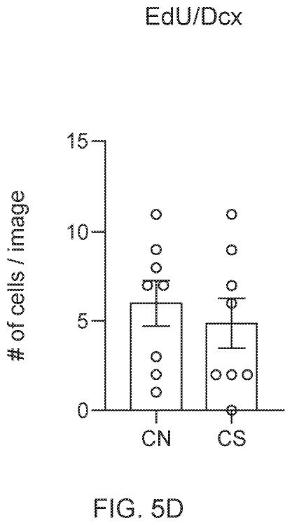

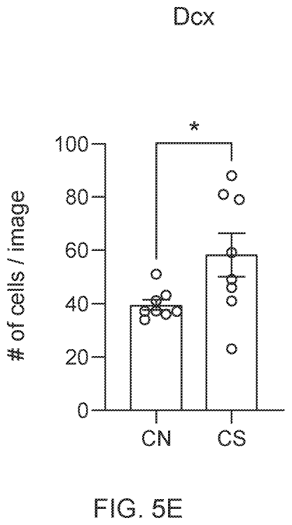

Proliferating cell population is majorly affected by chemotherapeutic agents in terms of DNA damage, and as a consequence, cancer patients often show reduced hippocampal neurogenesis, which is directly linked with cognitive functions. Tests were performed to determine if GENUS was able to restore impaired neurogenesis in chemo brain animals (FIGS. 5A, 5B). When proliferating cells were traced with EdU 4-6 days after the last cisplatin injection, Cis NS animals had significantly lower number of newborn neurons compared to PBS S animals, while Cis S animals had comparable number of newborn neurons with PBS treated animals (FIG. 5B). However, when proliferating cells were marked during the cisplatin injection, it was found that there were no difference in total number of EdU⁺Dcx⁺ cells between Cis NS and Cis S animals (FIG. 5D). Meanwhile, the total number of Dcx⁺ cell was significantly higher in Cis S animals, indicating the increased neurogenesis from GENUS treatment was happening outside the cisplatin treatment window.

GENUS Reduced Neuroinflammatory Signatures of Chemo Brain Mouse Model

Chronic inflammatory condition and reactive microglias as well as DNA damage have been pointed out to be underlying mechanisms of chemo brain in recent studies. Astrocyte and microglia, glial cells involved in inflammatory response, have been shown to both increase in cell number and acquire chronic reactive state in chemo brain mouse models. Recent findings using GENUS in other model animals systems have shown GENUS can modify the reactive state of both astrocyte and microglia. In line with other studies, it was found cisplatin treatment caused severe neuroinflammation with increased astrocyte and microglia signal in Cis NS hippocampus (FIGS. 2C, 2D). However, the gliosis was reversed in Cis S animals, showing comparable level of astrocyte and microglial signature with PBS controls (FIGS. 2D, 2E).

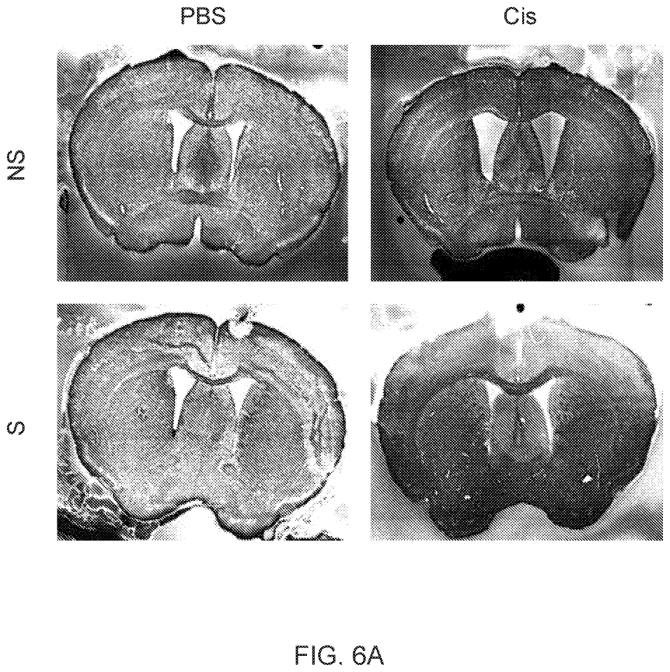

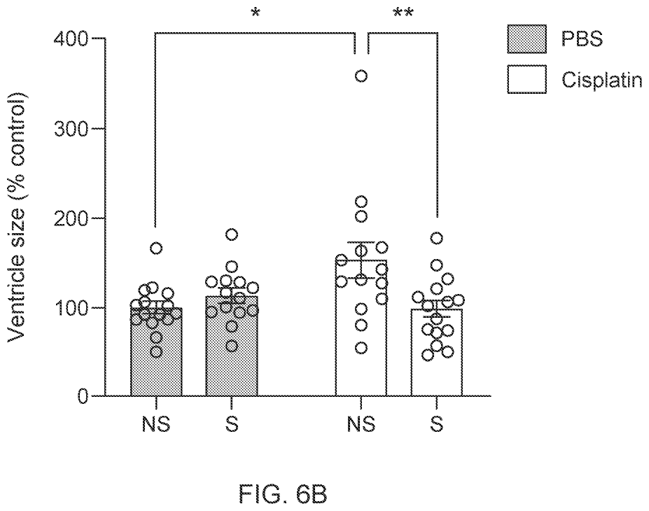

DNA damage and neuroinflammation has been repeatedly pointed out to be closely linked to neurodegenerative diseases. Tests were performed to determine if GENUS was able to protect the brain from neurodegeneration as well from cisplatin treatment. The size of ventricles were measured, as an enlarged cerebral ventricle is one of the common symptoms of neurodegeneration (FIGS. 6A, 6B). Cis NS animals displayed significantly enlarged lateral ventricles compared to PBS NS animals while Cis S animals on the other hand, had significantly smaller ventricles compared to Cis NS (FIG. 6B).

These findings show that GENUS protects the brain from overall toxic side effects of cisplatin such as DNA damage and chronic inflammation while boosting hippocampal neurogenesis up to comparable level with control animals.

GENUS Restored Impaired Cognitive Function in Chemo Brain Animals

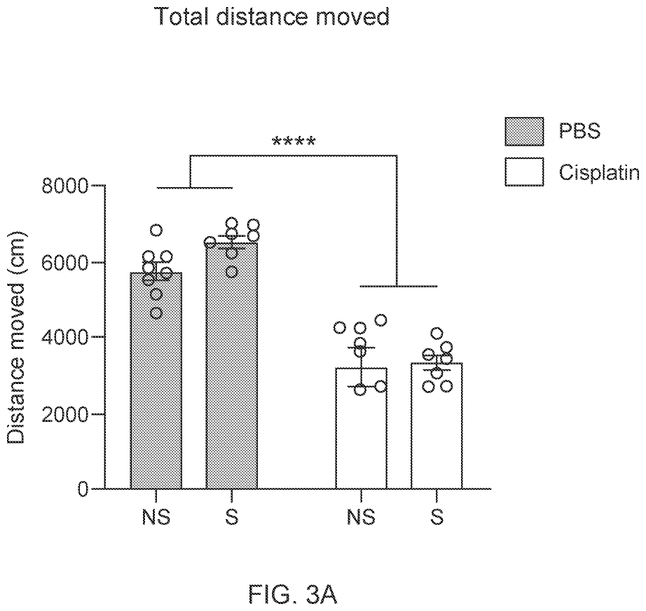

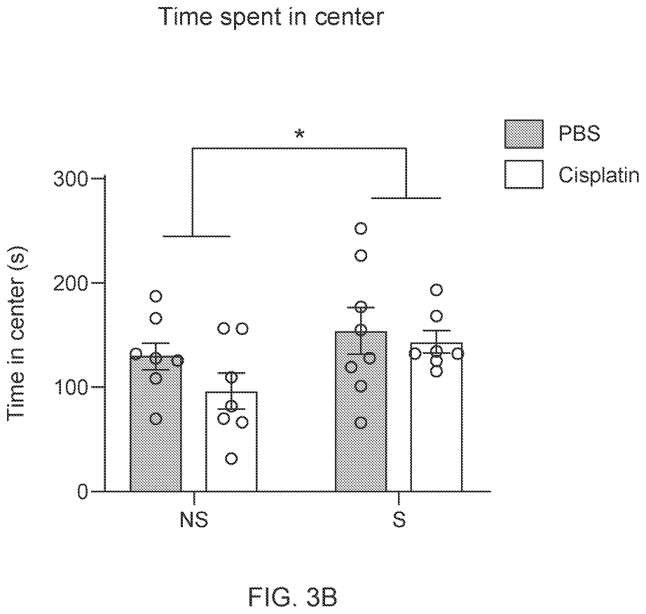

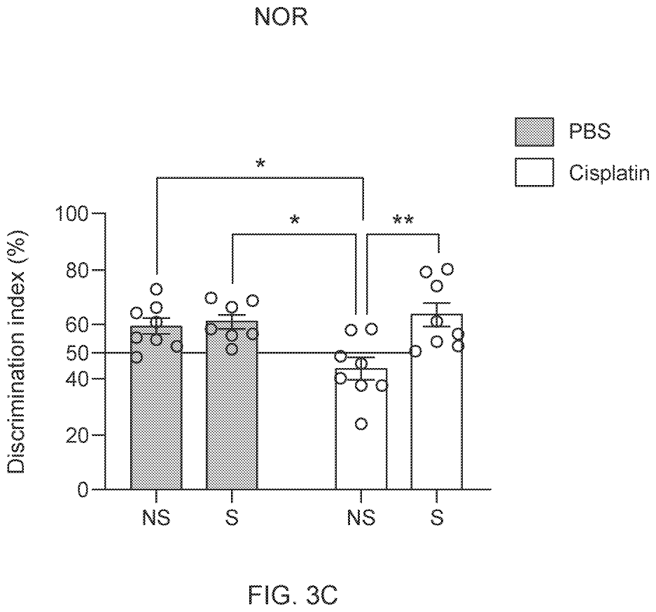

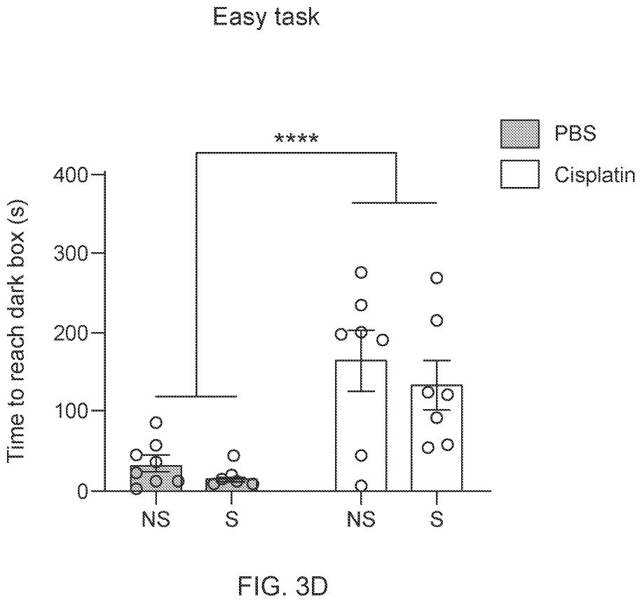

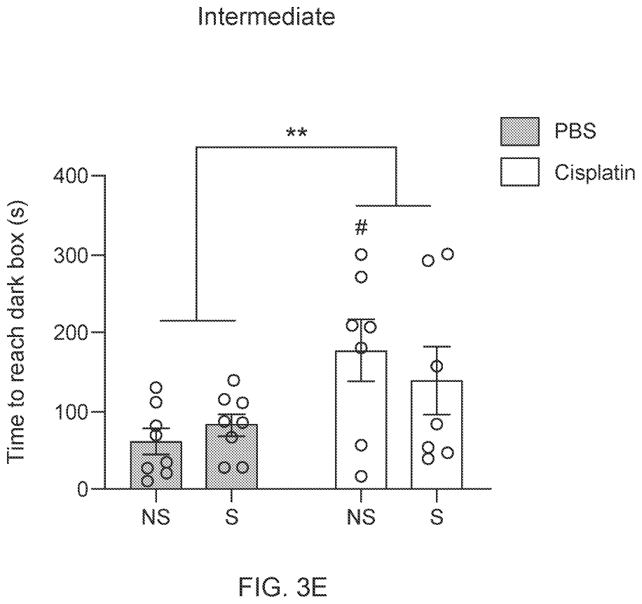

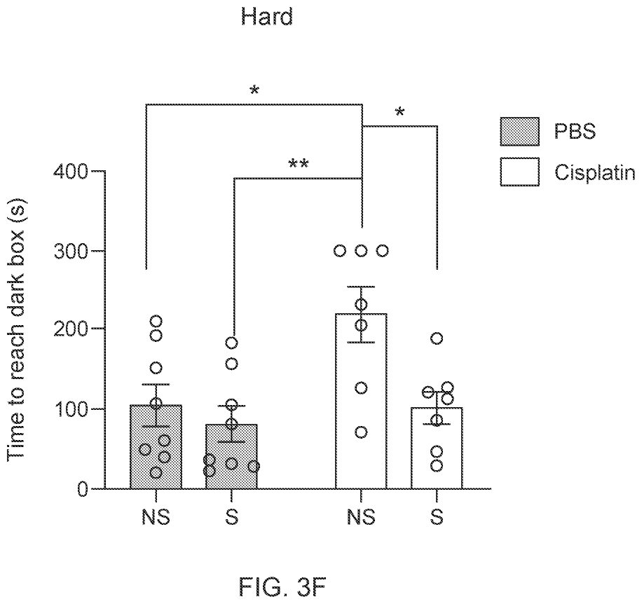

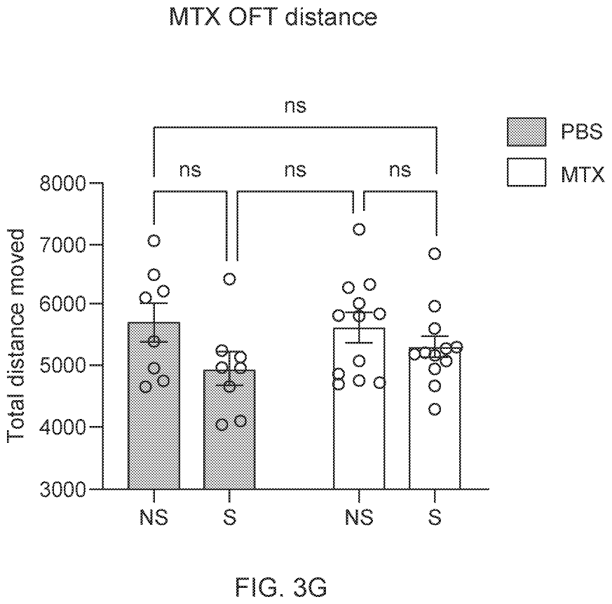

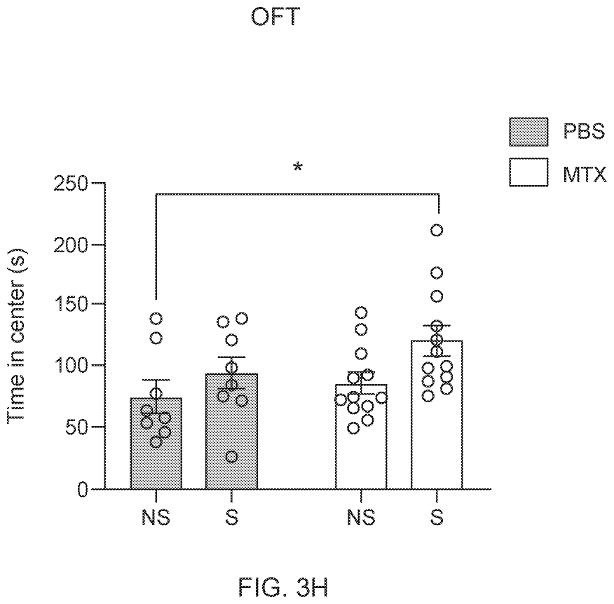

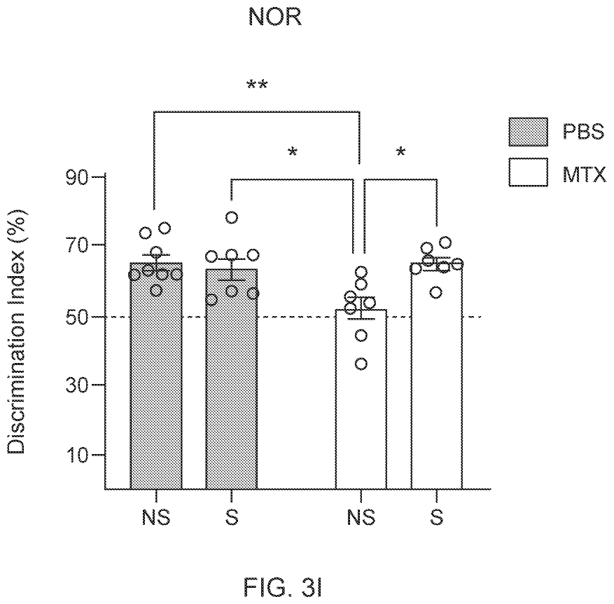

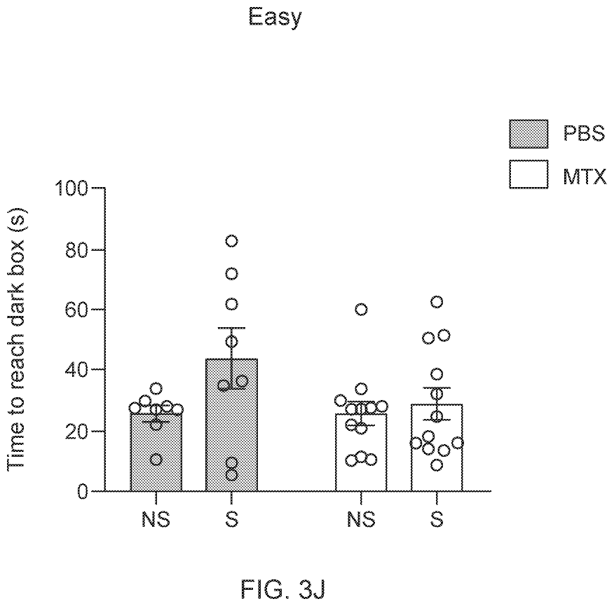

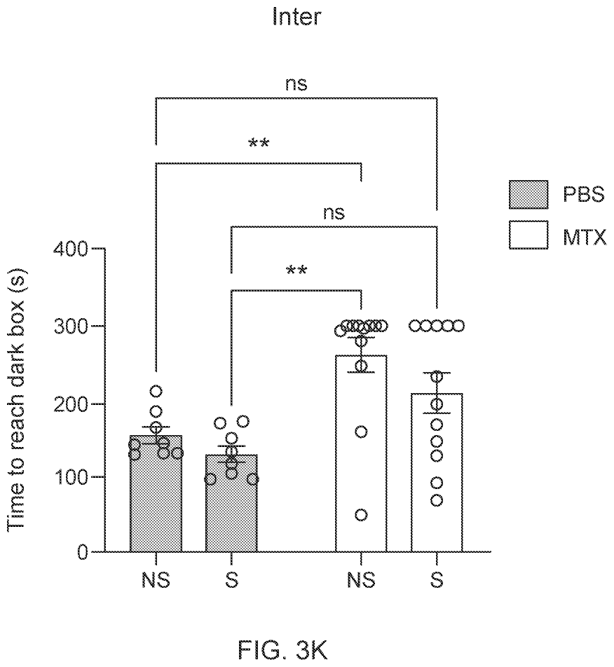

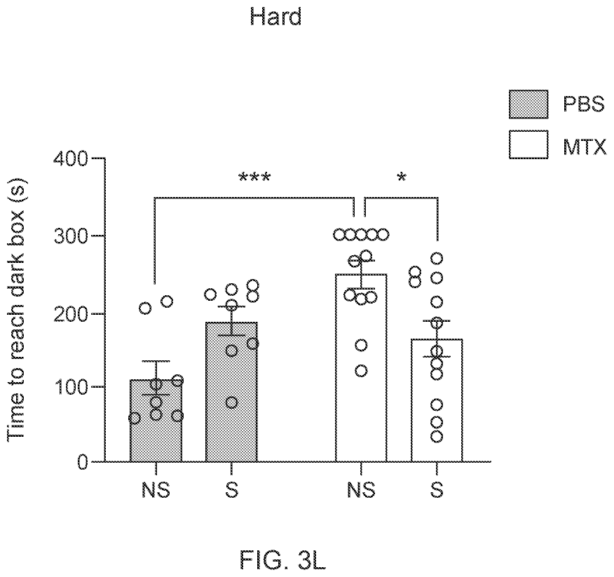

Next, it was tested if these molecular alterations actually lead to the restoration of cognitive function in chemo brain animals. An open field test (OFT) was performed prior to cognitive testing to see if cisplatin and GENUS had any effect on the locomotion and anxiety of animals (FIG. 3A, 3B). GENUS did not have any effect on animals locomotion, while cisplatin treatment significantly reduced the distance moved during 10 min OFT test (FIG. 3A). This may indicate a sign of peripheral neuropathy, which is one of the commonly observed side-effect in cisplatin treated patients in these animals. On the other hand, it was observed that GENUS, but not cisplatin treatment had significant effect on time spent in center area of the chamber (FIG. 3B), indicating reduced anxiety after GENUS treatment in both PBS and cisplatin treated animals.

Short term memory loss and attention deficit are the most common symptoms chemo brain patients encounter. A modified version of novel object recognition (NOR) test was used, which has been shown to be more attention and short term memory dependent. Here, it was found that Cis NS animals w spent comparable time exploring noble and familiar object, which means that they were not able to remember the previously encountered object while PBS NS and PBS S animals spent significantly more time exploring the novel object. Cis S animals also spent significantly longer time exploring the novel object, and the discrimination ratio was comparable to PBS control groups (FIG. 3C). Thereby, it was confirmed that GENUS is able to rescue cognitive impairment in chemo brain animal models induced with different chemotherapeutic agents.

Impaired problem-solving ability is another key symptoms that chemo brain patients suffer^{1,9}. Puzzle box test is a behavior test specializes in testing problem-solving ability in rodents with obstacles of various difficulties to enter a dark chamber innately preferred by rodents¹⁰, and have previously shown that cisplatin-induced chemo brain model animals show impaired performance¹. In this experimental cohort, three different obstacles with increasing difficulty (easy: open corridor, intermediate: sawdust, hard: tissue) to pass through was used to test if GENUS could rescue cisplatin-induced cognitive impairment (FIGS. 3D-3F). In both easy and intermediate tasks, cisplatin injected animals required longer time to enter the dark box (FIGS. 3D, 3E). In hard task, where animals had to pull out a tissue plugging the entrance to the dark box, cisplatin injected animals without GENUS treatment required longer time to reach the dark box, indicating that GENUS was able to rescue cisplatin-induced cognitive impairment (FIG. 3F).

GENUS was Effective in Methotrexate (MTX) Mouse Model

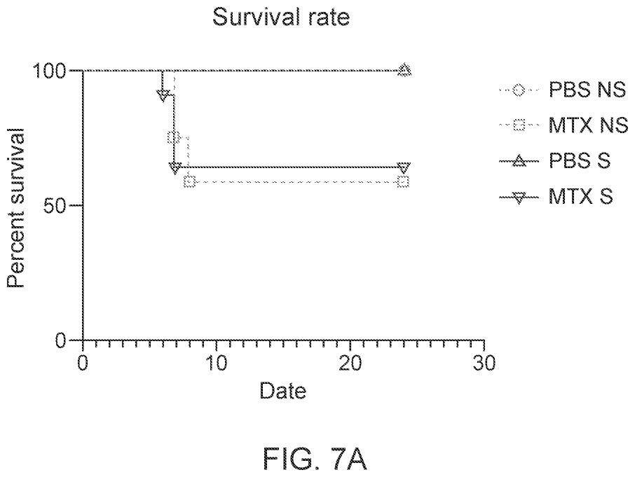

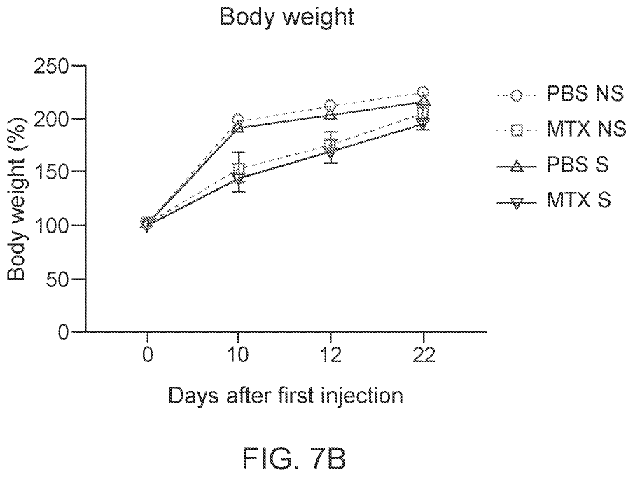

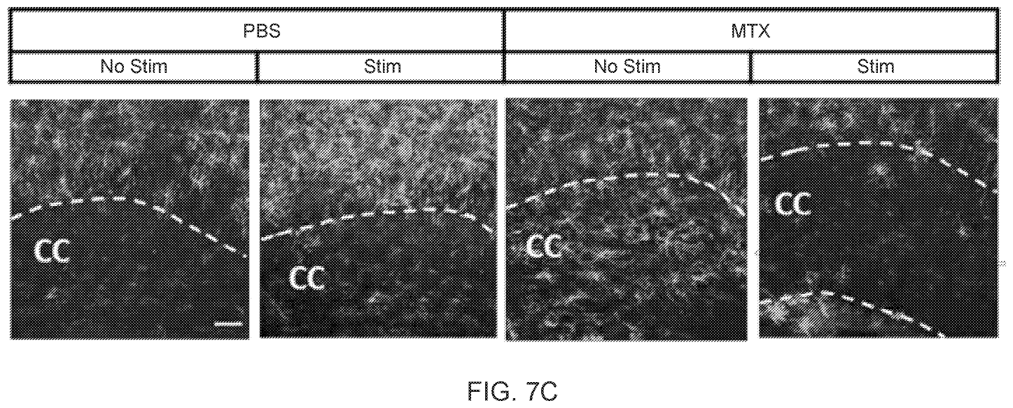

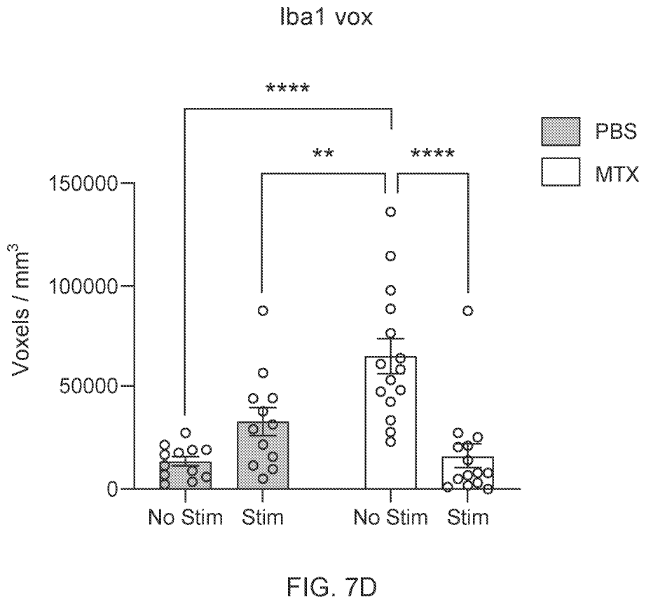

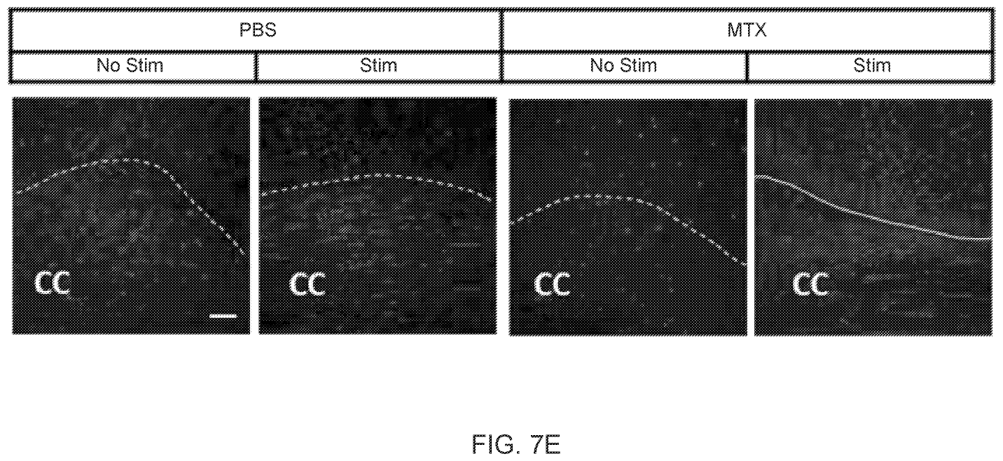

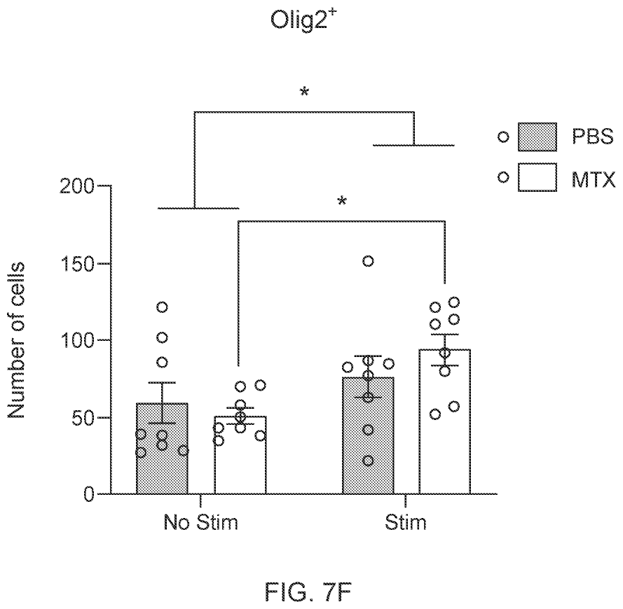

Finally, it was validated whether the effect of GENUS against cognitive impairment was specific to cisplatin or could be applied to a wider range of chemo brain caused by different chemotherapeutic agents. Another chemotherapeutic agent, methotrexate (MTX)-induced chemo brain animals were tested. As known in preceding reports, MTX treatment showed 40% lethality within 1 week after the first MTX injection, while none of the PBS injected animals were found dead (FIG. 7A). Since the animals were very young when they received their first injection, animals did not lose their body weight. Instead, MTX treatment significantly delayed body weight gain (FIG. 7B). As observed in cisplatin-induced chemo brain animal models, GENUS did not show any effect in terms of survival rate and body weight gain.

It was found MTX based chemo brain model animals show similar response to GENUS treatment based on histological features. It was found that the Iba1 signature was dramatically increased in corpus callosum of MTX treated animals and as seen in cisplatin-based chemo brain animals, Iba1 signature was significantly reduced in the corpus callosum after GENUS treatment (FIGS. 7C, 7D). Also, it was found that GENUS treatment increased Olig2⁺ positive cell count in the corpus callosum of these animals (FIGS. 7E, 7F).

Next, it was tested if GENUS could also rescue cognitive impairment in MTX-based chemo brain animals. Here, it was found that MTX-induced chemo brain model animals without GENUS spent comparable time exploring noble and familiar object, which means that they were not able to remember the previously encountered object. However, MTX-induced chemo brain model animals with GENUS spent significantly longer time exploring the novel object, and the discrimination ratio was comparable to PBS control groups (FIG. 3I). MTX S animals also showed enhanced performance in puzzle box test compared to MTX NS animals, especially in intermediate and hard tasks (FIGS. 3J-3L). Thereby, it was confirmed that GENUS is able to rescue cognitive impairment in chemo brain animal models induced with different chemotherapeutic agents.

Morris water maze test (MWM) was utilized to measure long-term spatial memory and motor function of animals. In the MWM test, all four groups (PBS NS, MTX NS, PBS S, MTXS) showed comparable performance in swimming speed, spatial learning and memory (FIGS. 1J-L). These results are in line with clinical reports that chemo brain patients do not show long term memory impairments.

Single Cell RNA Sequencing Revealed Transcriptional Alterations in Chemo Brain Animals Treated with GENUS

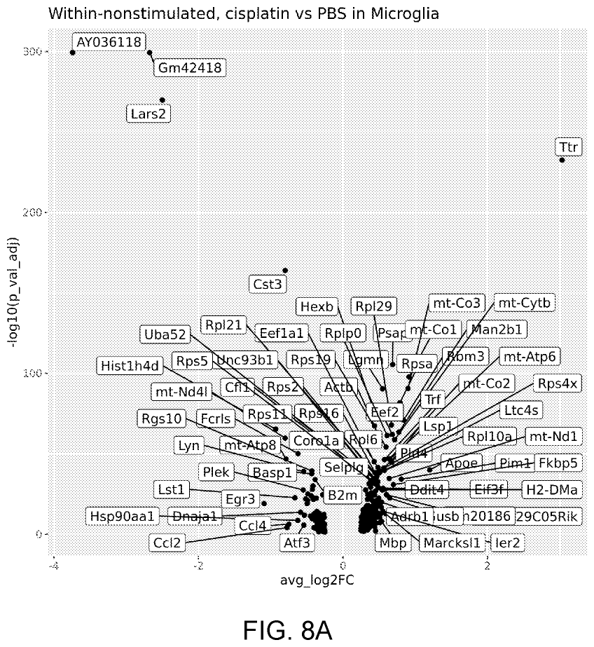

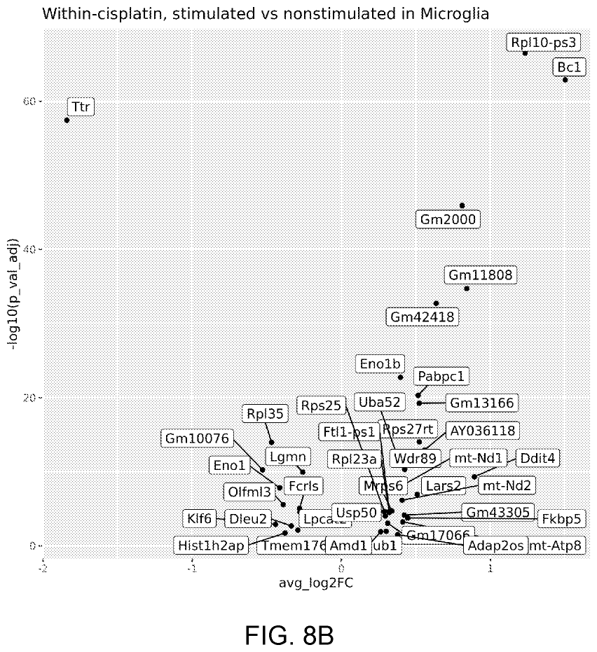

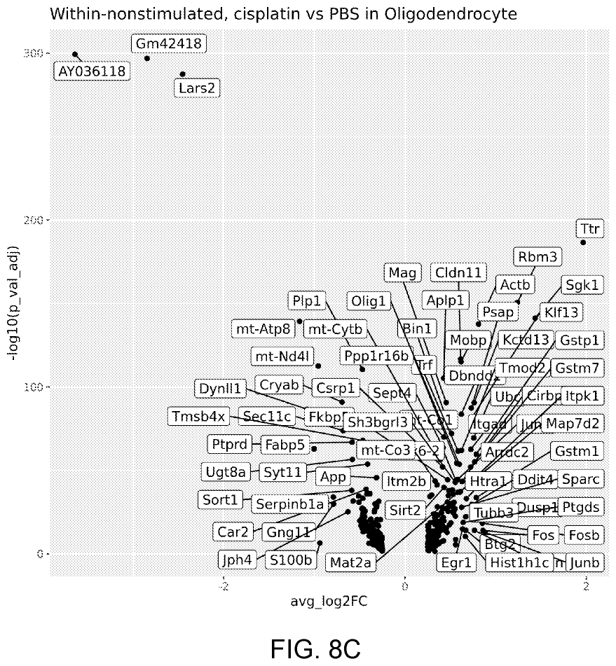

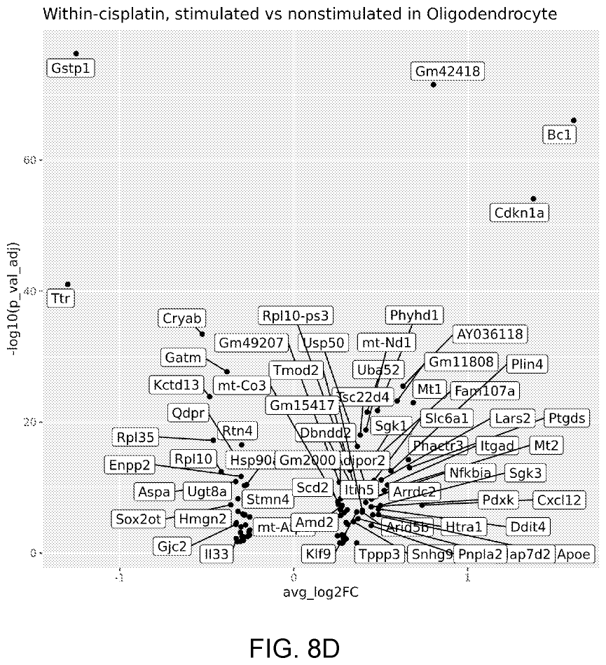

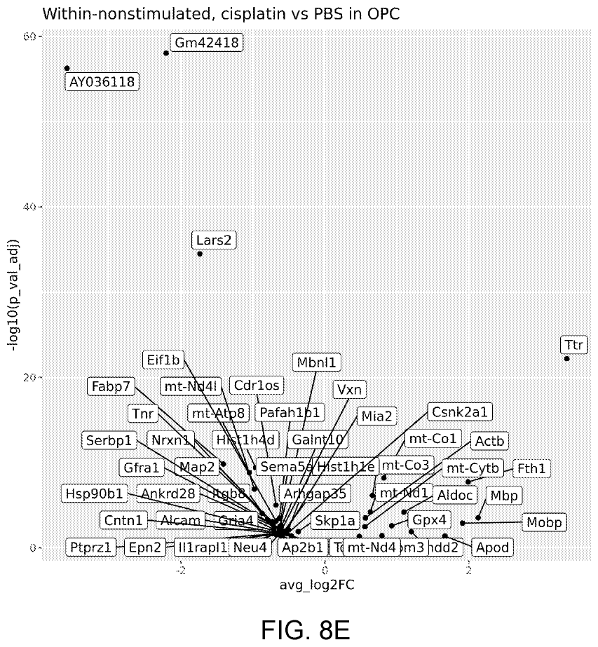

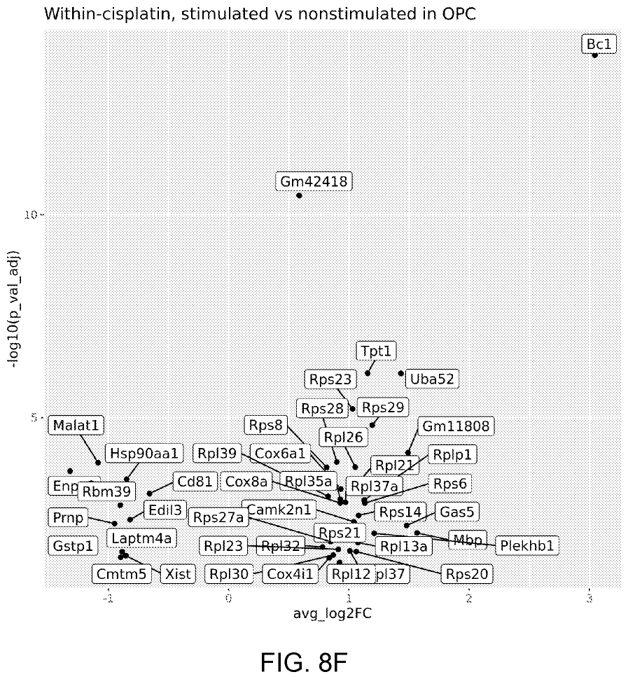

Single cell RNA sequencing was performed to investigate the effect of GENUS in transcriptional level in different cell types in the brain. Differential gene expression (DEG) analysis was performed with microglia (FIGS. 8A, 8B), oligodendrocyte (FIGS. 8C, 8D) and OPC (FIGS. 8E, 8F) cell clusters, comparing the effect of cisplatin treatment (PBS NS vs. Cis NS) and the effect of GENUS on chemo brain animals (Cis NS vs. Cis S).

Microglial cells showed upregulation of proinflammatory genes such as H2-DMa, Lsp1, Man2b1, Psap, Pim1, Apoe, Pld4 and Ddit4 in response to cisplatin treatment (FIG. 6A) while Cis S microglia had downregulated genes that trigger microglial inflammatory response such as Lgmn, Tmem176 and Dleu2 (FIG. 6B) compared to Cis NS, showing cisplatin induced inflammation, and showing the anti-inflammatory effect of GENUS.

Oligodendrocytes showed upregulation of genes those are expressed in white matter injury sites such as Fos, Fosb, and Ddit4 and also showed increased expression of myelin associated genes such as Mobp and Mag, which has been reported to occur in response to demyelination (FIG. 8C). Cis S oligodendrocytes had higher expression of genes involved in remyelination and oligodendrocyte maturation (Cdkn1a and Cxcl12) and genes those provide protective effect from oxidative stress or cell death (Bc1, Mt1 and Mt2) compared to Cis NS (FIG. 8D). These results may indicate the rescue of demyelination phenotype seen in Cis S animals might be the result of protection of oligodendrocytes from cisplatin induced oxidative stress and more active remyelination after the initial demyelination.

Oligodendrocyte precursor cells also showed higher expression of myeline associated genes such as Mbp and Mobp after cisplatin treatment, reflecting the response to demyelination (FIG. 8E). However, Cis NS OPCs showed downregulation of genes involved in OPC maturation such as Cntn1 and Tnr compared to Cis S OPCs (FIG. 8F). This may indicate that in Cis NS, OPCs are not properly maturating into oligodendrocytes resulting in insufficient myelinating cells and demyelination, while it is rescued with GENUS stimulation.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of treating cognitive impairment associated with chemotherapy treatment in a subject in need thereof, the method comprising:
non-invasively delivering a combined stimulus to the subject to invoke gamma entrainment in a brain of the subject, the combined stimulus including:
an auditory stimulus having a frequency of from about 20 Hz to about 60 Hz; and
a visual stimulus having a frequency of from about 20 Hz to about 60 Hz.

2. The method of claim 1, wherein the cognitive impairment includes short-term memory loss.

3. The method of claim 1, wherein the cognitive impairment includes attention deficit.

4. The method of claim 1, wherein the cognitive impairment includes increased anxiety.

5. The method of claim 1, wherein the cognitive impairment includes decreased problem solving ability.

6. The method of claim 1, wherein the auditory stimulus has a frequency of about 40 Hz, and wherein the visual stimulus has a frequency of about 40 Hz.

7. The method of claim 1, wherein the chemotherapy treatment includes administration of cisplatin or methotrexate.

11. The method of claim 1, wherein the chemotherapy treatment includes administration of one or more chemotherapy agents selected from the group consisting of an alkylating agent, a plant alkaloid, an antimetabolite, an anti-tumor antibiotic, a topoisomerase inhibitor, a mitotic inhibitor, and an antineoplastic.

12. The method of claim 1, wherein the chemotherapy treatment includes administration of one or more chemotherapy agents selected from the group consisting of 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abemaciclib, Abiraterone acetate, Abraxane, Acalabrutinib, Accutane, Actinomycin-D, Adcetris, Ado-Trastuzumab Emtansine, Adriamycin, Adrucil, Afatinib, Afmitor, Agrylin, Ala-Cort, Aldesleukin, Alemtuzumab, Alecensa, Alectinib, Alimta, Alitretinoin, Alkaban-AQ, Alkeran, All-transretinoic Acid, Alpelisib, Alpha Interferon, Altretamine, Alunbrig, Amethopterin, Amifostine, Aminoglutethimide, Anagrelide, Anandron, Anastrozole, Apalutamide, Arabinosylcytosine, Ara-C, Aranesp, Aredia, Arimidex, Aromasin, Arranon, Arsenic Trioxide, Arzerra, Asparaginase, Atezolizumab, Atra, Avastin, Avelumab, Axicabtagene Ciloleucel, Axitinib, Azacitidine, Balversa, Bavencio, Beg, Beleodaq, Belinostat, Bendamustine, Bendeka, Besponsa, Bevacizumab, Bexarotene, Bexxar, Bicalutamide, Bicnu, Binimetinib, Blenoxane, Bleomycin, Blinatumomab, Blincyto, Bortezomib, Bosulif, Bosutinib, Braftovi, Brentuximab Vedotin, Brigatinib, Busulfan, Busulfex, C225, Cabazitaxel, Cablivi, Cabozantinib, Calcium Leucovorin, Calquence, Campath, Camptosar, Camptothecin-11, Capecitabine, Caplacizumab-yhdp, Caprelsa, Carac, Carboplatin, Carfilzomib, Carmustine, Carmustine Wafer, Casodex, CCI-779, Ccnu, Cddp, Ceenu, Cemiplimab-rwlc, Ceritinib, Cerubidine, Cetuximab, Chlorambucil, Cisplatin, Citrovorum Factor, Cladribine, Clofarabine, Clolar, Cobimetinib, Cometriq, Cortisone, Cosmegen, Cotellic, Cpt-11, Crizotinib, Cyclophosphamide, Cyramza, Cytadren, Cytarabine, Cytarabine Liposomal, Cytosar-U, Cytoxan, Dabrafenib, Dacarbazine, Dacogen, Dacomitinib, Dactinomycin, Daratumumab, Darbepoetin Alfa, Darolutamide, Darzalex, Dasatinib, Daunomycin, Daunorubicin, Daunorubicin Cytarabine (Liposomal), daunorubicin-hydrochloride, Daunorubicin Liposomal, DaunoXome, Daurismo, Decadron, Decitabine, Degarelix, Delta-Cortef, Deltasone, Denileukin Diftitox, Denosumab, DepoCyt, Dexamethasone, Dexamethasone Acetate, Dexamethasone Sodium Phosphate, Dexasone, Dexrazoxane, Dhad, Die, Dinutuximab, Diodex, Docetaxel, Doxil, Doxorubicin, Doxorubicin Liposomal, Droxia, DTIC, Dtic-Dome, Duralone, Durvalumab, Eculizumab, Efudex, Ellence, Elotuzumab, Eloxatin, Elspar, Eltrombopag, Elzonris, Emapalumab-lzsg, Emcyt, Empliciti, Enasidenib, Encorafenib, Enhertu, Entrectinib, Enzalutamide, Epirubicin, Epoetin Alfa, Erbitux, Erdafitinib, Eribulin, Erivedge, Erleada, Erlotinib, Erwinia L-asparaginase, Estramustine, Ethyol, Etopophos, Etoposide, Etoposide Phosphate, Eulexin, Everolimus, Evista, Exemestane, Fam-Trastuzumab Deruxtecan-nxki, Fareston, Farydak, Faslodex, Fedratinib, Femara, Filgrastim, Firmagon, Floxuridine, Fludara, Fludarabine, Fluoroplex, Fluorouracil, Fluorouracil (cream), Fluoxymesterone, Flutamide, Folinic Acid, Folotyn, Fudr, Fulvestrant, G-Csf, Gamifant, Gazyva, Gefitinib, Gemcitabine, Gemtuzumab ozogamicin, Gemzar, Gilotrif, Gilteritinib, Glasdegib, Gleevec, Gleostine, Gliadel Wafer, Gm-Csf, Goserelin, Granix, Granulocyte—Colony Stimulating Factor, Granulocyte Macrophage Colony Stimulating Factor, Halaven, Halotestin, Herceptin, Herzuma, Hexadrol, Hexalen, Hexamethylmelamine, Hmm, Hycamtin, Hydrea, Hydrocort Acetate, Hydrocortisone, Hydrocortisone Sodium Phosphate, Hydrocortisone Sodium Succinate, Hydrocortone Phosphate, Hydroxyurea, Ibrance, Ibritumomab, Ibritumomab Tiuxetan, Ibrutinib, Iclusig, Idamycin, Idarubicin, Idelalisib, Idhifa, Ifex, IFN-alpha, Ifosfamide, IL-11, IL-2, Imbruvica, Imatinib Mesylate, Imfinzi, Imidazole Carboxamide, Imlygic, Inlyta, Inotuzumab Ozogamicin, INREBIC, Interferon-Alfa, Interferon Alfa-2b (PEG Conjugate), Interleukin-2, Interleukin-11, Intron A (interferon alfa-2b), Ipilimumab, Iressa, Irinotecan, Irinotecan (Liposomal), Isotretinoin, Istodax, Ivosidenib, Ixabepilone, Ixazomib, Ixempra, Jakafi, Jevtana, Kadcyla, Keytruda, Kidrolase, Kisqali, Kymriah, Kyprolis, Lanacort, Lanreotide, Lapatinib, Larotrectinib, Lartruvo, L-Asparaginase, Lbrance, Lcr, Lenalidomide, Lenvatinib, Lenvima, Letrozole, Leucovorin, Leukeran, Leukine, Leuprolide, Leurocri stine, Leustatin, Libtayo, Liposomal Ara-C, Liquid Pred, Lomustine, Lonsurf, Lorbrena, Lorlatinib, L-PAM, L-Sarcolysin, Lumoxiti, Lupron, Lupron Depot, Lynparza, Marqibo, Matulane, Maxidex, Mechlorethamine, Mechlorethamine Hydrochloride, Medralone, Medrol, Megace, Megestrol, Megestrol Acetate, Mekinist, Mektovi, Melphalan, Mercaptopurine, Mesna, Mesnex, Methotrexate, Methotrexate Sodium, Methylprednisolone, Meticorten, Midostaurin, Mitomycin, Mitomycin-C, Mitoxantrone, Mogamulizumab KPKC, Moxetumomab, M-Prednisol, MTC, MTX, Mustargen, Mustine, Mutamycin, Mvasi, Myleran, Mylocel, Mylotarg, Navelbine, Necitumumab, Nelarabine, Neosar, Neratinib, Nerlynx, Neulasta, Neumega, Neupogen, Neulasta Onpro, Nexavar, Nilandron, Nilotinib, Nilutamide, Ninlaro, Nipent, Niraparib, Nitrogen Mustard, Nivolumab, Nolvadex, Novantrone, Nplate, Nubeqa, Obinutuzumab, Octreotide, Octreotide Acetate, Odomzo, Ofatumumab, Olaparib, Olaratumab, Omacetaxine, Oncospar, Oncovin, Onivyde, Ontak, Onxal, Opdivo, Oprelvekin, Orapred, Orasone, Osimertinib, Otrexup, Oxaliplatin, Paclitaxel, Paclitaxel Protein-bound, Palbociclib, Pamidronate, Panitumumab, Panobinostat, Panretin, Paraplatin, Pazopanib, Pediapred, Peg Interferon, Pegaspargase, Pegfilgrastim, Peg-Intron, PEG-L-asparaginase, Pembrolizumab, Pemetrexed, Pentostatin, Perj eta, Pertuzumab, Phenylalanine Mustard, Piqray, Platinol, Platinol-AQ, Pomalidomide, Pomalyst, Ponatinib, Portrazza, Poteligeo, Pralatrexate, Prednisolone, Prednisone, Prelone, Procarbazine, Procrit, Proleukin, Prolia, Prolifeprospan 20 with Carmustine Implant, Promacta, Provenge, Purinethol, Radium 223 Dichloride, Raloxifene, Ramucirumab, Rasuvo, Regorafenib, Revlimid, Rheumatrex, Ribociclib, Rituxan, Rituxan Hycela, Rituximab, Rituximab Hyalurodinase, Roferon-A (Interferon Alfa-2a), Romidepsin, Romiplostim, Rozlytrek, Rubex, Rubidomycin Hydrochloride, Rubraca, Rucaparib, Ruxolitinib, Rydapt, Sandostatin, Sandostatin LAR, Sargramostim, Siltuximab, Sipuleucel-T, Soliris, Solu-Cortef, Solu-Medrol, Somatuline, Sonidegib, Sorafenib, Sprycel, Sti-571, Stivarga, Streptozocin, SU11248, Sunitinib, Sutent, Sylvant, Synribo, Tafmlar, Tagraxofusp-erzs, Tagrisso, Talimogene Laherparepvec, Talazoparib, Talzenna, Tamoxifen, Tarceva, Targretin, Tasigna, Taxol, Taxotere, Tecentriq, Temodar, Temozolomide, Temsirolimus, Teniposide, Tespa, Thalidomide, Thalomid, TheraCys, Thioguanine, Thioguanine Tabloid, Thiophosphoamide, Thioplex, Thiotepa, Tibsovo, Tice, Tisagenlecleucel, Toposar, Topotecan, Toremifene, Torisel, Tositumomab, Trabectedin, Trametinib, Trastuzumab, Treanda, Trelstar, Tretinoin, Trexall, Trifluridine/Tipiricil, Triptorelin pamoate, Trisenox, Truxima, Tspa, T-VEC, Tykerb, Unituxin, Valrubicin, Valstar, Vandetanib, VCR, Vectibix, Velban, Velcade, Vemurafenib, Venclexta, Venetoclax, VePesid, Verzenio, Vesanoid, Viadur, Vidaza, Vinblastine, Vinblastine Sulfate, Vincasar Pfs, Vincristine, Vincristine Liposomal, Vinorelbine, Vinorelbine Tartrate, Vismodegib, Vitrakvi, Vizimpro, Vlb, VM-26, Vorinostat, Votrient, VP-16, Vumon, Vyxeos, Xalkori Capsules, Xeloda, Xgeva, Xofigo, Xospata, Xtandi, Yervoy, Yescarta, Yondelis, Zaltrap, Zanosar, Zarxio, Zejula, Zelboraf, Zevalin, Zinecard, Ziv-aflibercept, Zoladex, Zoledronic Acid, Zolinza, Zometa, Zydelig, Zykadia, and Zytiga.

13. The method of claim 1, wherein the chemotherapy treatment is for treating, in the subject, one or more of a carcinoma, a sarcoma, a melanoma, a lymphoma, or a leukemia.

14. The method of claim 1, wherein the chemotherapy treatment is for treating, in the subject, one or more of Acute Lymphoblastic Leuknemia (ALL), Acute Myeloid Leukemia (AML), Cancer in Adolescents, Adrenocortical Carcinoma, Kaposi Sarcoma (Soft Tissue Sarcoma), AIDS-Related Lymphoma (Lymphoma), Primary CNS Lymphoma (Lymphoma), Anal Cancer, Appendix Cancer, Childhood Astrocytoma, Childhood Atypical Teratoid/Rhabdoid Tumor of the Central Nervous System, Basal Cell Carcinoma of the Skin, Bile Duct Cancer, Bladder Cancer, Bone Cancer, Ewing Sarcoma, Osteosarcoma, Malignant Fibrous Histiocytoma, Brain Tumors, Breast Cancer, Bronchial Tumor (Lung Cancer), Burkitt Lymphoma, Carcinoid Tumor (Gastrointestinal), Carcinoma of Unknown Primary, Childhood Cardiac (Heart) Tumor, Childhood Atypical Teratoid/Rhabdoid Tumor, Medulloblastoma, CNS Embryonal Tumors, Childhood (Brain Cancer) Childhood Germ Cell Tumor, Primary CNS Lymphoma, Cervical Cancer, Childhood Cancers, Unusual Cancers of Childhood, Unusual, Cholangiocarcinoma, Childhood Chordoma, Chronic Lymphocytic Leukemia (CLL), Chronic Myelogenous Leukemia (CML), Chronic Myeloproliferative Neoplasm, Colorectal Cancer, Childhood Craniopharyngioma, Cutaneous T-Cell Lymphoma, Ductal Carcinoma In Situ (DCIS), Childhood Embryonal Tumors, Childhood Medulloblastoma and Other Central Nervous System Cancers, Endometrial Cancer, Childhood Ependymoma, Esophageal Cancer, Esthesioneuroblastoma (Head and Neck Cancer), Ewing Sarcoma (Bone Cancer), Childhood Extracranial Germ Cell Tumor, Extragonadal Germ Cell Tumor, Intraocular Melanoma, Retinoblastoma, Fallopian Tube Cancer, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumor (GIST) (Soft Tissue Sarcoma), Childhood Central Nervous System Germ Cell Tumor, Childhood Extracranial Germ Cell Tumor, Extragonadal Germ Cell Tumor, Ovarian Germ Cell Tumor, Testicular Cancer, Gestational Trophoblastic Disease, Hairy Cell Leukemia, Head and Neck Cancer, Childhood Heart Tumors, Hepatocellular (Liver) Cancer, Langerhans Cell Histiocytosis, Hodgkin Lymphoma, Hypopharyngeal Cancer, Intraocular Melanoma, Islet Cell Tumor, Pancreatic Neuroendocrine Tumor, Kaposi Sarcoma (Soft Tissue Sarcoma), Kidney (Renal Cell) Cancer, Langerhans Cell Histiocytosis, Laryngeal Cancer, Leukemia, Lip and Oral Cavity Cancer, Liver Cancer, Lung Cancer (Non-Small Cell, Small Cell, Pleuropulmonary Blastoma, and/or Tracheobronchial Tumor), Lymphoma, Male Breast Cancer, Malignant Fibrous Histiocytoma of Bone and Osteosarcoma, Melanoma, Intraocular Melanoma (Eye), Merkel Cell Carcinoma, Malignant Mesothelioma, Metastatic Cancer, Metastatic Squamous Neck Cancer with Occult Primary, Midline Tract Carcinoma With NUT Gene Changes, Mouth Cancer, Multiple Endocrine Neoplasia Syndromes, Multiple Myeloma/Plasma Cell Neoplasm, Mycosis Fungoides, Myelodysplastic Syndrome, Myelodysplastic/Myeloproliferative Neoplasm, Chronic Myelogenous Leukemia, Acute Myeloid Leukemia, Chronic Myeloproliferative Neoplasm, Nasal Cavity and Paranasal Sinus Cancer, Nasopharyngeal Cancer, Neuroblastoma, Non-Hodgkin Lymphoma, Non-Small Cell Lung Cancer, Oral Cancer, Lip and Oral Cavity Cancer, Oropharyngeal Cancer, Osteosarcoma and Malignant Fibrous Histiocytoma of Bone, Ovarian Cancer, Pancreatic Cancer, Pancreatic Neuroendocrine Tumors (Islet Cell Tumors), Papillomatosis (Childhood Laryngeal), Paraganglioma, Paranasal Sinus and Nasal Cavity Cancer, Parathyroid Cancer, Penile Cancer, Pharyngeal Cancer, Pheochromocytoma Pituitary Tumor, Plasma Cell Neoplasm/Multiple Myeloma, Pleuropulmonary Blastoma, Pregnancy and Breast Cancer, Primary Central Nervous System (CNS) Lymphoma, Primary Peritoneal Cancer, Prostate Cancer, Rectal Cancer, Recurrent Cancer, Renal Cell (Kidney) Cancer, Retinoblastoma, Childhood Rhabdomyosarcoma, Salivary Gland Cancer, Childhood Rhabdomyosarcoma, Childhood Vascular Tumors, Ewing Sarcoma, Kaposi Sarcoma, Osteosarcoma, Soft Tissue Sarcoma, Uterine Sarcoma, Sezary Syndrome, Skin Cancer, Small Cell Lung Cancer, Small Intestine Cancer, Soft Tissue Sarcoma, quamous Cell Carcinoma of the Skin, Metastatic Squamous Neck Cancer with Occult Primary, Stomach (Gastric) Cancer, Cutaneous T-Cell Lymphoma, Testicular Cancer, Throat Cancer, Thymoma, Thymic Carcinoma, Thyroid Cancer, Tracheobronchial Tumor, Transitional Cell Cancer of the Renal Pelvis and Ureter, Carcinoma of Unknown Primary, Unusual Cancers of Childhood, Transitional Cell Cancer of Ureter and Renal Pelvis, Urethral Cancer, Endometrial Uterine Cancer, Uterine Sarcoma, Vaginal Cancer, Vascular Tumor, Vulvar Cancer, Wilms Tumor and Other Childhood Kidney Tumors, or Cancer in Young Adults.

15-16. (canceled)

17. The method of claim 1, wherein the auditory stimulus includes a 40 Hz clicking sound having a 50% duty cycle and wherein the visual stimulus includes a 40 Hz flickering light having a 50% duty cycle.

18. (canceled)

19. The method of claim 1, wherein the combined stimulus is non-invasively administered for at least about 1 hour.

20. (canceled)

21. The method of claim 1, wherein the combined stimulus is non-invasively administered with no waiting period after the chemotherapy treatment.

22. The method of claim 1, wherein the combined auditory and visual stimuli is non-invasively administered from about 1 hour to about 12 hours after the chemotherapy treatment, and wherein the combined auditory and visual stimuli is non-invasively administered the same day as the chemotherapy treatment.

23. (canceled)

24. A method of treating cognitive impairment associated with chemotherapy treatment in a subject in need thereof, the method comprising:
delivering a stimulus to the subject to invoke gamma entrainment in a brain of the subject, the stimulus having a frequency of from about 20 Hz to about 60 Hz.

25. The method of claim 24, wherein the stimulus has a frequency from about 35 Hz to about 45 Hz.

26. A method of reducing neuroinflammation in a brain region of a subject, the neuroinflammation associated with chemotherapy treatment in the subject in need thereof, the method comprising:
delivering a stimulus to the subject to invoke gamma entrainment in a brain of the subject, the stimulus having a frequency of from about 20 Hz to about 60 Hz.

27. The method of claim 26, wherein the stimulus has a frequency from about 35 Hz to about 45 Hz.

28. The method of claim 26, wherein the brain region is the hippocampus of the subject, and wherein the method comprises delivering the stimulus to the subject to invoke gamma entrainment in the brain of the subject to reduce a number of microglia in the hippocampus of the subject.

29. The method of claim 26, wherein the brain region is the hippocampus of the subject, and wherein the method comprises delivering the stimulus to the subject to invoke gamma entrainment in the brain of the subject to reduce a number of astrocytes in the hippocampus of the subject.

30-37. (canceled)
